(19) United States
(12) Patent Application Publication
(10) Pub. No.: US 2009/0126032 A1
(43) Pub. Date: May 14, 2009

(54) Method to produce cloned embryos and adults from cultured cells

(75) Inventors: Anthony C.F. Perry, New York, NY (US); Peter Mombaerts, New York, NY (US); Teruhiko Wakayama, Honolulu, HI (US)

Correspondence Address: ROPES & GRAY LLP, PATENT DOCKETING 39/41, ONE INTERNATIONAL PLACE, BOSTON, MA, 02110-2624, US

(73) Assignee: Advanced Cell Technology, Inc.

(21) Appl. No.: 12/221,060

(22) Filed: Jul. 29, 2008

Related U.S. Application Data
(63) Continuation of application No. 10/168,341, filed on Oct. 16, 2002, now abandoned.
(60) Provisional application No. 60/172,683, filed on Dec. 20, 1999.

(51) Int. Cl. A01K 67/033; C12N 15/89; C12N 5/00

(52) U.S. Cl. 800/8; 800/24; 435/325

(57) ABSTRACT

A nuclear transfer method is provided wherein nuclear DNA in whole or part is injected into enucleated oocytes. The method is suitable for different donor cells, and preferably ES cells.

### TECHNICAL FIELD OF THE INVENTION

A method is described to clone embryos and live offspring from cells cultured in vitro. Preferably, the cells are established cell lines, and more preferably, they are embryonic stem (ES) cells. Also disclosed are cell lines derived from clonally-derived embryos. We describe different embodiments of the invention that show that the method is not critically dependent upon cell cycle stage or genomic complement of the nucleus donor cell. The method has potential utility in the production of clonally-derived tissues and organisms with or without targeted mutations. This potential is all the greater given that prior art does not allow a single cell from an established line to program full embryonic development to term.

### BACKGROUND OF THE INVENTION

Mammals have previously been cloned by effecting the fusion of a nucleus donor cell with an enucleated oocyte (Willadsen, Nature 320, 63 [1986]). This method was originally described in sheep (Willadsen, Nature 320, 63 [1986]) and has subsequently been further applied to quiescent somatic cells of sheep (Campbell, et al., Nature 380, 64 [1996]; Schnieke et al., Science 278, 2130 [1997]; Wilmut, et al., Nature 385, 810 [1997]), and to proliferating somatic cells of cattle (Cibelli, et al., Science 280, 1256 [1997]; Kato, et al., Science 282, 2095 [1998]; Renard, et al., Lancet 353, 1489 [1999]; Wells, et al., Biol. Reprod. 60, 996 [1999]) and goats (Baguisi, et al., Nature Biotech. 17, 456 [1999]). The nucleus donor cells described in these reports are freshly isolated from an animal or from short-term primary cell cultures. The sheep named ‘Dolly’ was reportedly cloned using this method from a mammary-derived cell of unknown identity (Wilmut, et al., Nature 385, 810 [1997]).

More recently, a distinctive method of cloning has been developed in which the nucleus of a donor cell from the tissue of adult mammal is first selected and then microinjected into an enucleated oocyte (Wakayama, et al., Nature 394, 369 [1998]). The microinjection method can be used to produce viable embryos, live offspring and healthy adult animals which can optionally be genetically engineered. Applications of this method of nuclear transfer have enabled the cloning of live-born offspring using adult-derived cumulus cells to clone females (Wakayama, et al., Nature 394, 369 [1998]) and tail-derived cells to clone males (Wakayama & Yanagimachi, Nature Genet. 22, 127 [1999]). The clonal provenance of these animals has been rigorously verified by phenotypic and genomic analyses (Wakayama, et al., Nature 394, 369 [1998]).

Both cell fusion and microinjection methods to date suffer from the drawback that they describe the use of freshly isolated cells or cells from primary, often ill-defined cell cultures as nucleus donors. This is due in part to epigenetic instabilities in cultured cells (Dean, et al., Development 125, 2273 [1998]). Any cloning method that circumvented these problems would permit cells to be engineered in vitro before they were used as nucleus donors in the cloning process. This would have great utility: it would, for example, allow for the generation of clones containing genomically targeted mutations and permit long-term storage of clonal progenitor cells.

Cultured embryonic stem (ES) cells (eg., ES cell lines) are derived from the inner cell mass (ICM) of blastocysts and exhibit unusual karyotypic and cytogenetic stability in vitro (Evans, et al., Nature 292, 154 [1981]; Martin, et al., Proc. Natl. Acad. Sci. USA 78, 7634 (1981); Hogan, et al., Manipulating the mouse embryo. 2nd ed. [Cold Spring Harbor Laboratory Press], pp 173-181 [1994]). Mouse ES cells exhibit developmental pluripotency: when transferred into mouse embryos they can generate chimaeric offspring containing an ES cell contribution that is apparently unrestricted in terms of cell type (Hogan, et al., Manipulating the mouse embryo. 2nd ed. [Cold Spring Harbor Laboratory Press], pp 173-181 [1994]; Bradley, et al., Nature 309, 255 [1994]). However, for ES cells to contribute fully to the development of an individual, they must be accompanied by heterologous cells from a developing embryo (hence, the embryo is chimaeric). The heterologous cells are from diploid (Bradley, et al., Nature 309, 255 [1984]; Hooper, et al., Nature 326, 292 [1987]) or tetraploid (Nagy, et al., Development 110, 815 [1990]; Nagy, et al., Proc. Natl. Acad. Sci. USA 90, 8424 [1993]; Zang, et al., Mech. Dev. 62, 137 [1997]) embryos. Unless they are rescued by the heterologous cells of a developing embryo, it is not possible for ES cells to program full-term embryonic development. This is a major drawback for the use of ES cells since they cannot direct embryonic development capable of going toward full-term development; offspring generated from them have therefore previously necessarily been chimaeric. This necessitates lengthy breeding programs to obtain descendents derived exclusively from the ES cells.

ES cells can be used to introduce targeted genomic alterations into an animal. Gene targeting in ES cells has been widely used to create manifold strains of mice with targeted mutations (Capecchi, Science 244, 1288 [1989]); Ramirez-Solis, et al., Mets. Enzymol. 225, 855-878 [1993]). The introduction of targeted mutations utilizes homologous recombination to ‘knock out’ or ‘knock in’ targeted segments of the genome to replace them with an incoming gene. The phenotypic effect of the mutation may be tailored by the choice of the incoming gene, which may completely alter the phenotype, or alter it subtly. Cloning animals from ES cells could combine the advantages of gene targeting and animal cloning to facilitate the production of gene-targeted animals. If nuclei from ES cell lines—even after prolonged in vitro culture—could be used to produce viable, fertile cloned animals, they would be a prime choice for engineering the mammalian genome through cloning. However, some previous difficulties have included the development of suitable culturing and selective procedures to efficiently allow for selection of ES cells in targeted procedures rather than random DNA modifications.

Prior art has not yet demonstrated that any cultured ES cell lines, or ES cell-like cell lines or other established cell lines can direct full development following nuclear transfer, even though nuclear transfer has been used to produce sheep, cattle and goats. For instance, Campbell, et al. (Nature 380, 64 [1996]) have reported the cloning of sheep by nuclear transfer from short-term cultured, embryonically-derived epithelial cells via a cell fusion method; however, these cells expressed markers associated with differentiation and cellular commitment, and were therefore clearly not ES cells.

Stice, et al. (WO 95/17500) have reported the production of bovine embryos by membrane fusion nuclear transfer with contemporaneously-derived, low passage ES cell-like cells. Stice, et al. provide no examples of the success of their nuclear transfer method in producing offspring (live or still-born), from these or any other ES cell-like cells, because all pregnancies aborted prior to 60 days gestation; the longest pregnancy was 55 days, with an average gestation period of 280 days in cows.

Tsunoda and Kato (J. Reprod. Fert. 98, 537 [1993]) reported the development in vitro to two-cell, four-cell, morula and blastocyst stages, of enucleated mouse eggs that were fused (by Sendai virus and electrofusion) to ES cell nuclei from lines that had been passaged 11-20 times. However, no live fetuses were obtained after the transfer of the resulting embryos to surrogate mothers.

In marked contrast, the method of the invention now disclosed permits the generation of live offspring from the nucleus of a single, cultured cell.

### SUMMARY OF THE INVENTION

The invention described herein provides a solution to these short-comings. It provides a method for the clonal propagation of differentiated cells (for example, in the form of a whole animal) from a single, reconstituted cell. A donor nucleus is typically inserted into an enucleated recipient cell, e.g., an oocyte or blastomere, and generates a reconstituted cell. Development of the resulting reconstituted cell is initiated and cultivated. Hence, in related embodiments, the invention provides for (i) the clonal derivation of an embryo from an ES cell by inserting the nuclear contents of the ES cell into the cytoplasm of an enucleated oocyte and allowing the reconstituted cell to differentiate, and (ii) cultured cells or an animal produced by this method.

In one embodiment, differentiation of the resulting reconstituted cell is along one or more specified pathways resulting in the production of a variety of different cell types. In another embodiment, development of the resulting reconstituted cell is into an embryo that in turn develops into a viable, live-born offspring. As used herein, the term ‘nucleus’ is intended to encompass the entire nucleus or a portion thereof, wherein the nuclear contents include at least the minimum material able to direct development in a cell lacking any other non-mitochondrial genome. The resulting tissue is clonally derived from the cell that provided the nucleus for injection into the enucleated oocyte (the nucleus donor); where the procedure results in offspring, the offspring is a clone derived from the nucleus donor cell.

Hence, the invention provides methods for cloning an animal from an ES cell line by inserting the nucleus of a cell from a cultured ES cell line into an enucleated oocyte. The nucleus donor may be from a well-established cell line, or it may be from a freshly-derived cell line. In some animals, e.g. mammals, the majority of established ES cell lines will be male-derived; that is, they possess an XY karyotype. By contrast, in avians, the majority of established ES cell lines will be female-derived; that is, they possess an XX karyotype. Whole animal clones derived from such XY cell lines thus reflect this provenance and are male. Accordingly, in an embodiment in which nucleus donors are from female-derived cell lines, whole animal clones with an XX karyotype are produced and are female, and the opposite is true with animals derived from ES cells of the XY karyotype.

In a further embodiment, cells used in the method of the invention are derived from species other than the mouse, including but not limited to those in the groups of primates, ovines, bovines, porcines, ursines, felines, caprines, canines, equines, cetids and murines and other rodents. In a favored embodiment, ES cell-like cells are derived from the ICM of blastocysts from these species.

In a further embodiment, the ES cells from which the nucleus donor cell is to be sourced, is established just prior to its use. In a favored embodiment, ES cells are genetically modified prior to their use in the production of clonally-derived cells, such as cloned animals.

Cells reconstituted following ES cell nuclear transfer may develop into a blastocyst following culture in vitro or such development may be effected in vivo, e.g. with porcines. In one embodiment, the blastocyst may be transferred to a suitable surrogate foster mother to produce a cloned animal arisine from the reconstituted cell.

In another embodiment, a morula or blastocyst clonally derived by the method of the invention may, in turn, be aggregated (or injected) with ES cells derived from the culture used initially to provide the nucleus donor that generated the clonally-derived embryo. This results in a embryo whose cells arise partly from the cloned embryo and partly from the injected/aggregated cells of the cultured ES cells. These methods of aggregation and injection are well-established amongst those skilled in the art and are the same in principle as the ones used to produce chimaeric embryos in standard gene targeting protocols (Hogan, et al., Manipulating the mouse embryo. 2nd ed. [Cold Spring Harbor Laboratory Press], pp. 189-216 [1994]; Joyner [ed], Gene targeting. [Oxford University Press], pp. 107-146 [1993]). However, the embryos generated in the method of the invention now disclosed are not chimeric with respect to their nuclear genomes, since resulting live offspring are derived from genetically identical ES cells. This embodiment of the method enhances the efficiency of production of cloned live offspring from ES cells.

In a further embodiment, the morula or blastocyst clonally derived by the method of the invention may be utilized as a source of stem cells such as cells of the inner cell mass (ICM) in blastocysts. Such cells can be caused to differentiate along prescribed pathways according to methods known by those skilled in the art. This embodiment of the invention therefore produces differentiated cells of a given type, from any cultured population of nucleus donor cells. Cell types that can be generated by this method include, without limitation, cell types located in widespread anatomical locations, such as epithelial cells, blood cells and fibroblasts and the like, and cells exhibiting greater anatomical restriction, such as cardiomyocytes, hematopoietic cells, neuronal cells, glial cells, keratinocytes, and the like.

We demonstrate herein the production of live offspring cloned from the nuclei of ES cells from established ES cell lines derived from F1 and inbred mouse strains. In one embodiment of the invention, cloned live offspring are produced from ES cell nuclei that are ‘2C’; that is, they possess the diploid complement of genomic DNA, as seen in pre-5-phase cells at the G0- or G1-phases of the cell cycle.

In another embodiment of the invention, the donor ES cell nucleus is ‘2-4C’. Although for most of the life of a dividing cell, it contains 2C DNA represented in 2n chromosomes, there is a period following S-phase of the cell cycle, wherein the chromosome number remains unaltered but the DNA content has been doubled by a duplicative round of DNA synthesis; hence such cells are 2n, but 4C, until the separation of the sister chromatids of bivalent chromosomes at telophase. The use of 4C nuclei in one embodiment of the invention, produces live, cloned offspring. This demonstrates that it is not necessary for (ES) cells to be in the G0- or G1-phases of the cell cycle in order for their nuclei to direct development of any cell type.

In one embodiment, the ES cell nucleus donor has been genetically altered to harbor a desired mutation. Hence, an animal or population of cells cloned by the method of the invention from the genetically altered ES cell will possess the mutation. The genetic alterations(s) in the ES cell may be the result of a non-directed mutation, of mutagenesis by exposure to mutagenic agents, or of the introduction into the cell of an exogenous nucleic acid or nucleic acid derivative by known methods (such as electroporation, retroviral infection, and the like). More preferably, the ES cell used as the nucleus donor has been genetically altered by gene targeting, such that part or all of one or more specific genes have been modified in a precise and controlled manner.

Thus, the invention provides a method for producing cloned, genetically altered live offspring in one generation from cell lines (including, but not restricted to ES cell lines) that can be genetically manipulated and characterized in vitro prior to nuclear transfer. The invention method thus enhances the speed and efficiency by which gene-targeted animals are produced from the corresponding cell lines.

### BRIEF DESCRIPTION OF THE DRAWINGS

FIG. 1 is a schematic representation of the cloning procedure of the present invention, and is explained in the text.

FIG. 2 is a table containing the results of an experiment wherein enucleated oocytes received E14 nuclei but were not subjected to an activating stimulus.

FIG. 3 is a table containing the results of an experiment wherein enucleated oocytes received E14 nuclei, and were activated with strontium ions after nuclear transfer.

FIG. 4 is a table summarizing the results of experiments in which 1765 oocytes were reconstructed using nuclei from E14 cells of different sizes and grown with different concentrations of FCS.

FIG. 5 is a table containing results of an experiment wherein 1087 nuclear transfers were effected with the cell line R1, which was derived from the F1 hybrid, 129/SV x 129/SV-CP.

### DETAILED DESCRIPTION OF THE INVENTION

The instant invention discloses that viable, live born offspring may be obtained by inserting nuclear components (including the chromosomes) of an embryonic stem (ES) cell into an enucleated oocyte and facilitating the development of the resulting reconstituted cell to term. ES cells may be cultured or cryopreserved long-term prior to use in nuclear transfer. Isolation, culture and manipulation of mouse ES cells—including gene targeting by homologous recombination—is described in: Hogan, et al., Manipulating the mouse embryo. 2nd ed. (Cold Spring Harbor Laboratory Press), pp. 253-290 (1994). Methods for establishing either ES cells or cells that resemble ES cells (ES cell-like cells) have been described for cattle (Cibelli, et al., Theriogenology 47, 241 [1997]), hamster, (Doetschman, et al., Dev. Biol. 127, 224 [1988]), human (Thomson, et al., Science 282, 1145 [1998]) and rabbit (Schoonjans, et al., Mol. Reprod. Dev. 45, 439 [1996]).

Offspring derived from ES cell nuclei according to the invention are genomic clones in which the chromosomes of every cell of the offspring are derived from those of the original nucleus donor ES cell.

Preferably, the ES cell is from an ES cell line whose stem cell properties have been demonstrated via germ line contribution and transmission in chimaeric offspring following standard blastocyst injection procedures known to those of ordinary skill in the art (Bradley, et al., Nature, 309, 255 [1984]; Hogan, et al., Manipulating the mouse embryo. 2nd ed. [Cold Spring Harbor Laboratory Press], pp. 196-204 [1994]). This process commonly involves the injection of ES cells into the cavities of blastocysts arising from fertilization. In this cellular context, ES cells are able to participate in development to form a chimaeric animal that is derived partly from the host blastocyst and partly from the injected ES cell(s). ES cells can give rise to somatic tissue in the chimaera and are capable of contributing to all cell types, including the germ line of the chimaera. The ability of ES cells to contribute to an extensive range of cell types is called ‘pluripotency’. Demonstration of ES pluripotency in germ line transmission is limited to mice and cattle, although there is no known reason to believe that the phenomenon is restricted to these species. ES cell lines are considered to provide a powerful tool for studies of mammalian genetics, developmental biology and medicine.

ES cells may be from an established ES cell line. Such ES cell lines are well known and include, but are not limited to, those derived from F1 hybrid strains and inbred mouse strains. Examples of ES cell lines derived from F1 hybrid strains include R1 (Nagy, A. et al., Proc. Nail. Acad. Sci. USA, 90, 8424 [1993]) (see Example 2). Examples of ES cell lines derived from inbred strains include the 129/01a-derived male lines E14 (Hooper, M., et al., Nature 326, 292 [1987]) (available from the American Type Culture Collection, Bethesda, Md. [ATCC] number CRL-11632), D3 (ATCC number CRL-1934) and AB1 and AB2.2, commercially available from Lexicon Genetics.

In addition to mouse ES cell lines, ES cell-like cells have been obtained from cattle (Cibelli, et al., Theriogenology 47, 241 [1997]), hamster, (Doetschman, et al., Dev. Biol. 127, 224 [1988]), human (Thomson, et al., Science 282, 1145 [1998]) and rabbit (Schoonjans et al., Mol. Reprod. Dev. 45, 439 [1996]). Technical barriers thwart the application of the same rigorous criteria to ES cells from these animals as for mice, namely that they are extensively pluripotent and capable of contributing to most or all cell fates including the germ line. It might be expected that experimentally substantiated ES cell lines fulfilling all defining criteria for ES cells will be demonstrated for species other than the mouse.

Cells other than ES cells (or ICM-derived cells) might be cultured in vitro sufficient for genome manipulation and/or use as nucleus donors in a whole animal cloning procedure. Such cell-types are not species-restricted and may be exemplified by lines of human fibroblasts, porcine embryonic germ (EG) cells (REF), and mouse embryonal carcinoma (EC) cells (Stewart, & Mintz, J. Exp. Zool. 224, 465 [1982]; Hogan, et al., Manipulating the mouse embryo. 2nd ed. [Cold Spring Harbor Laboratory Press], p 92 [1994]). The variety of cells amenable to long-term culture and genetic manipulation in vitro is likely to increase; all such cells are potential nucleus donors in the method of the invention.

ES cell lines can be demonstrably engineered with respect to their genomes. Methods for achieving this are now well established and there are manifold reports in the literature of engineering ES cell lines so that they have a given genetic (and often corresponding phenotypic) trait (Mombaerts, et al., Proc. Nad. Acad. Sci. USA, 88, 3084 [1991]; Mombaerts, et al., Nature 360, 225 [1992]; Itohara, et al., Cell 72, 337 [1993]). This is, in turn, achieved by introducing recombinant DNA by, for example, electroporation or lipofection. Mutant ES cells may also arise spontaneously in culture and may be enriched in the presence of selective culture media. For example, it was reported that variant ES cells deficient in hypoxanthine guanine phosphoribosyl transferase (HPRT) were selected in culture by their resistance to the purine analogue 6-thioguanine, and that these mutant ES cells were used to produce germ line chimaeras resulting in male offspring deficient for HPRT (Hooper, et al., Nature 326, 292 [1987]).

A key feature of ES cell technologies is that they permit the targeted alteration of DNA sequences in the context of an entire genome. This relies on a phenomenon called homologous recombination, in which DNA sequences align with their complementary (matching, or near-identical) genomic sequences within a cell. The complementary sequences are called homologous sequences. The sequences may then undergo an exchange reaction (crossing over) which results in sequences of the incoming DNA effectively replacing those resident on the chromosome. If the incoming sequence is near-identical to its genomic counterpart, or if it is interspersed with additional unrelated sequences, this replacement results in the targeted introduction of a new sequence. The replacement utilizes cellular enzymes whose normal role is thought to be in DNA repair and maintenance. For reasons unknown at present, ES cells are a rich source of such enzymes and are the only well-characterized mammalian cell known readily to support homologous (ie., targeted) recombination. Gene targeting, then, results in the production of an ES cell in which one or more specific loci are modified in a precisely prescribed manner. Examples of gene targeting include the production of ‘knock out’ and ‘knock in’ mice using incoming DNA sequences that are part of relatively short (<˜25 kilobase pairs [kbp]) recombinant DNA segments. It is anticipated that ES cell-like cells may also be gene-targeted using techniques similar to those used for gene targeting ES cells.

Current methods using gene-targeted ES cells lines to produce genetically altered mice involve the injection or aggregation of engineered ES cells respectively with, or into, morulae (approximately 8 cells) or blastocysts (upwards of 16 cells). Upon implantation, such embryos may give rise to chimeric parent (F0) animals, whose subsequent breeding with wild-type animals results in germ line transmission of the ES cell-derived genome at variable frequencies (often equal to zero). Any first generation (F1) offspring to which the targeted gene modification has been transmitted are identified phenotypically (for example, by their coat color) and by analysis of their genomic DNA (Joyner [ed], Gene targeting. [Oxford University Press], pp. 52-59 [1993]; Hogan, et al., Manipulating the mouse embryo. 2nd ed. [Cold Spring Harbor Laboratory Press], pp. 291-324 [1994]).

Breeding of F1 heterozygotes is usually necessary and in some cases generates second generation (F2) animals homozygous for the mutation. Thus, the current procedure for producing animals homozygous for a gene-targeted mutation involves at least three generations of animals. In mice, this requires of the order of at least six months to establish pure-breeding lines that are homozygous for a given mutant allele. However, for the majority of mammals, including commercially valuable breeds, which have a much longer gestation/maturation period, the time required to produce pure-breeding lines would be far longer. For example, in cattle, three generations would require at least 3×280 days, or approximately 2.3 years.

Since ES cell lines are clonal (in the sense of cell cloning, not whole animal cloning), their use in whole animal cloning enables the relatively rapid production of identical animals in essentially unlimited numbers. It would therefore be possible to produce a large number of identical animals by using a single population of ES cells as nucleus donors to generate a corresponding number of reconstituted cells that could be brought to develop to term. The proliferation of near-identical, genetically engineered animals is expected to provide enormous benefits to human and veterinary medicine and farming. For example, genetically altered animals (including larger animals) can act as living pharmaceutical ‘factories’ by producing valuable pharmaceutical agents in their milk or other fluids or tissues, usually secretory tissues. This production method is sometimes referred to as ‘pharming’.

The production of large numbers of identical research animals, such as mice, guinea pigs, rats, and hamsters is also desirable because of its utility in drug discovery and screening. The availability of colonies of near-identical mice is highly beneficial in the analysis of, for example, development, human disease, and in the testing of new pharmaceuticals; inherent variability between individuals is minimized, facilitating comparative studies.

The present invention describes a method for generating differentiated cell population, such as clones of animals from cultured cells, such as ES cells, by nuclear transfer. In the method, clonally derived cells develop from an enucleated oocyte that has received the nucleus (or a portion thereof, including at least the chromosomes) of an ES cell, for example, from an established ES cell line. In one embodiment of the invention, cloned mice may be produced following microinjection of the nucleus of an ES cell into an enucleated oocyte by the method of the invention. In a further embodiment, the ES cell nucleus donor may be from the ES cell line, E14. Offspring that have been cloned from ES cells may be recognized by their coat color several days postnatally, reflecting the phenotype of the mouse strain from which the nucleus donor cell line was derived. Many ES cell lines presently available are derived from the 129 mouse strain, 129/Sv, which was derived by Dr. Leroy Stevens at the Jackson Laboratory.

The invention is applicable to cloning of all animals from which ES cells can or might be isolated and cultured to form ES cell lines, including amphibians, fish, birds (e.g., domestic chickens, turkeys, geese, and the like) and mammals, such as primates, ovines, bovines, porcines, ursines, felines, canines, equines, caprines, murines and the like.

An embodiment of the method of the invention includes the steps of (i) allowing the ES nucleus to be in contact with the cytoplasm of the enucleated oocyte for a period of time (e.g., up to about 6 hours) after its insenion into the oocyte, but prior to the activation of development, and (ii) activating the reconstituted cell to initiate development.

In one embodiment, a donor nucleus having a 2C genomic complement is employed. Where the nucleus donor is 2C, activation is preferably in the presence of an inhibitor of microtubule and/or microfilament assembly in order to suppress the extrusion of chromosomes in a pseudo-polar body. Where, for example, a 4C donor nucleus is employed, the reconstituted cell may be incubated for up to approximately 6 hours prior to activation in the absence of the microtubule/microfilament inhibitor; in such cases, a pseudo-polar body is extruded such that the ploidy of the reconstituted cell may be restored to 2n. (Modal 2n ploidy is normally a prerequisite to direct embryonic development beyond gastrulation.)

In a preferred embodiment of the invention, the ES cell nucleus is inserted into the cytoplasm of the enucleated oocyte by microinjection and, more preferably, by piezo-electrically-actuated microinjection. The use of a piezo-electric micromanipulator enables the harvesting and injection of the donor nucleus from the ES cell to be performed with a single needle. Moreover, enucleation of the oocyte and injection of the donor ES cell nucleus can be performed quickly, efficiently and with reduced consequent trauma to the oocyte compared to previously reported methods (eg., fusing of the donor cell and oocyte mediated by fusion-promoting chemicals, by an electrical discharge or by a fusogenic virus).

The method of introducing nuclear material by microinjection is distinct from introducing nuclear material by cell fusion, both temporally and topologically. In the microinjection method of the current invention, first the plasma membrane of the donor ES cell is punctured and subsequently, the plasma membrane of the enucleated oocyte is punctured. Hence, extraction of the nucleus (Or a portion thereof including at least the chromosomes) from the donor cell is temporally separated from delivery of that nucleus into the recipient cell. This spatial and temporal separation of the isolation and delivery of nuclear contents is not a feature of cell fusion, in which two cells are juxtaposed and then in a single step, caused to fuse.

Furthermore, the spatiotemporal separation of nucleus removal and introduction in the method of the invention, allows controlled introduction of material in addition to the nucleus. The facility to remove extraneous material (such as cytoplasm and nucleoplasm) and to introduce additional materials or reagents may be highly desirable. For example the additive(s) may favorably influence subsequent development. Such a reagent may comprise an antibody, a pharmacological signal transduction inhibitor, or combinations thereof, wherein the antibody and/or the inhibitor are directed against and/or inhibit the action of proteins or other molecules that have a negative regulatory role in cell division or embryonic development. The reagent may include a nucleic acid sequence, such as a recombinant plasmid or a transforming vector construct, that may be expressed during development of the embryo to encode proteins that have a potential positive effect on development and/or a nucleic acid sequence that becomes the introduction of a reagent into a cell may take place prior to, during, or after the combining of a nucleus with an enucleated oocyte.

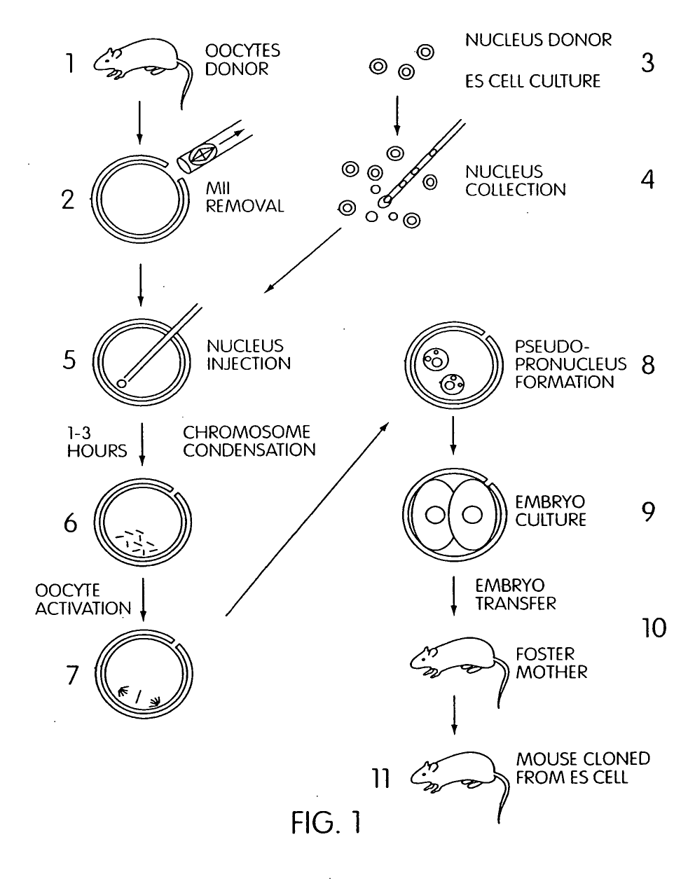

Steps and substeps of one embodiment of the method of the invention for clonally deriving differentiated cell populations by nuclear transfer from cultured ES cells are illustrated in FIG. 1.

In summary, oocytes are harvested (1) from an oocyte donor animal, preferably metaphase I stage oocytes, and the metaphase II (mII) plate (containing the mII chromosomes) of each is removed (2) to form an enucleated oocyte (devoid of maternally-derived chromosomes). Recipient oocytes may be matured in vitro by known procedures or in vivo as has been described by other researchers. Healthy-looking ES cells are chosen (3,4) from an in vitro culture containing cells which may be of small (typically 10 μm) or large (typically 18 μm) diameter, as accommodated by different embodiments of the current invention. A single nucleus is injected (5) into the cytoplasm of an enucleated oocyte. The nucleus is allowed to reside within the cytoplasm of the enucleated oocyte (6) for up to 6 hours. In one embodiment, this period is a minimal period of approximately 0-5 min. In a preferred embodiment, the period is 1-3 hours.

The oocyte is then activated in the presence or absence of an inhibitor of microtubule and/or microfilament assembly (7), depending on the ploidy or genomic equivalence of the incoming nucleus as reflected in part by the cell cycle stage of the donor nucleus at the time of transfer. The mitotic cell cycle ensures that following a duplicative round of DNA replication, cells that are actively dividing donate equal genetic material to two daughter cells. DNA synthesis does not occur throughout the cell cycle but is restricted to one part of it: the synthesis phase, S-phase. This is followed by a gap phase, G2-phase, during which the cell further prepares for division before entering metaphase (M-phase). Nascent daughter cells are thence delivered into another gap phase, the G1-phase. Apparently, certain non-dividing cells, for example terminally differentiated cells in vivo, are suspended at this stage in the cycle—the stage which corresponds in dividing cells to G1-phase and which precedes the S-phase. Such cells are frequently referred to as ‘resting’, and to have exited from the cell cycle to enter the G0-phase. The nuclei of cells in G0- or G1-phases of the cell cycle are diploid, with 2n chromosomes corresponding in this case to a 2C DNA content; they have two copies of each morphologically distinct autosome (non-X, non-Y); and depending upon species, either an XX (female) or XY pair. The nuclei of cells in the G2-phase of the cell cycle, having undergone a round of DNA replication, are still 2n with respect to chromosome number, but now have a 4C DNA content. During S-phase, DNA in each of the two copies of each of the distinct chromosomes is replicated, but the copies (univalent sister chromatids) are tethered at the centromere of each chromosome. Within a non-synchronously dividing ES cell culture one may expect, by definition, all stages of the cell cycle to be represented. Consequently, ES cell cultures contain a mixture of cells reflected by a range of diameters; this range may be from approximately 10 μm to approximately 18 μm. Relatively small cells (approximately 10 μm in diameter) are likely diploid (2n) and 2C with respect to their genomic DNA, since these cells have relatively recently divided with relatively little subsequent increase in cytoplasmic volume. Cells tending towards the largest size (approximately 18 μm in diameter) are more likely to have advanced beyond S-phase.

Where the ES cell donor nucleus is diploid and 2C, the reconstituted cell is activated (7) in the presence of an inhibitor of cytokinesis following nuclear transfer. This suppresses the formation of a pseudo-polar body and prevents chromosome loss, consequently sustaining the 2n ploidy of the reconstituted cell. Where the nucleus is considered likely to be post S-phase (because it is within a larger cell) the oocyte is activated in the absence of the cytokinesis inhibitor so that formation of a pseudo-polar body can concomitantly reduce the ploidy of the oocyte to 2n, 2C. During the activation period, formation of pseudo-pronuclei may be observed.

The concentration of fetal calf serum (FCS) in the ES nucleus donor cell culture medium may be varied over a wide range; the FCS concentration is not believed to exert significant influence on the ability of nuclei from the cultured ES cells to support development of cloned live offspring by the method of the invention.

Following transfer of the nuclei of either small or large cells, reconstructed oocytes forming pseudo-pronuclei (8) are transferred to fresh media for embryo culture for 1 to approximately 3.5 days (9). Following culture, embryos may be transferred (10) to surrogate mothers to permit the development and the birth (11) of live offspring. Alternatively, the embryo generated in (9) may be used as a source of ICM cells in the subsequent derivation of ES cell-like cell cultures.

Thus, one embodiment of the method of the present invention describes the cloning of a manual comprising the steps of: (a) collecting all or part of the nucleus of a cell such as an ES cell, including at least the chromosomes; (b) inserting it into an enucleated oocyte; (c) allowing the reconstituted cell to develop into an embryo; and (d) allowing the embryo to develop into a fetus and subsequently a live offspring, or causing the cells of the embryo to be cultured in vitro. Each of these steps is described below in detail, with an ES cell nucleus donor as the exemplar.

The ES cell nucleus (or nuclear constituents containing the chromosomes) may be collected from an ES cell that has a genomic DNA complement of 2-4C as described above. Preferably, the ES cell nucleus is inserted into the cytoplasm of the enucleated oocyte. The insertion of the nucleus is preferably accomplished by microinjection and, more preferably, by piezo electrically-actuated microinjection. In further embodiments, the nucleus may be introduced by allowing the nucleus donor cell to fuse with the recipient, enucleated oocyte (Willadsen, Nature 320, 63 [1986]).

Activation of the reconstituted cell may take place prior to, during, or after the insertion of the ES cell nucleus. In one embodiment, the activation step takes place from zero to about six hours after insertion of the ES cell nucleus. During the time preceding activation, the nucleus is in contact with the resident cytoplasm of the mII oocyte (potentially modified by incoming components). Activation may be achieved by various means including, but not limited to electroactivation, or exposure to ethanol, sperm cytoplasmic factors, oocyte receptor lit and peptide mimetics, pharmacological stimulators of Ca²⁺ release (e.g., caffeine), Ca²⁺ ionophores (e.g., A2318, ionomycin), modulators of phosphoprotein signaling, inhibitors of protein synthesis, and the like, or combinations thereof. In one embodiment of the invention, the activation is achieved by exposing the cell to strontium ions (Sr²⁺).

The activation of reconstituted cells that had been injected with nuclei containing 2C DNA is preferably accomplished by exposure to an inhibitor of microtubule and/or microfilament assembly to prevent the formation of a polar body (see below). This favors retention of all the chromosomes from the donor nucleus within the reconstituted cell. Reconstituted cells that had received 2-4C nuclei are preferably activated in the absence of such an inhibitor in order to allow the formation of a pseudo-polar body, thereby reducing the genomic complement to 2C. In one embodiment, the 2C genomic complement corresponds to 2n chromosomes.

The step of allowing the embryo to develop may include the substep of transferring the embryo to a recipient surrogate mother wherein the embryo develops into a viable fetus (that is, an embryo that successfully implants sufficient for normal development to term). The embryo may be transferred at any stage of in vitro development, from two-cell to morula/blastocyst, as known to those skilled in the art.

The first ten steps of an additional embodiment of the invention produce a cloned morula or blastocyst (embryo) according to steps (1) to (10) in FIG. 1. In one embodiment, subsequent to this, and prior to transferring the cloned embryo to a surrogate recipient female, at least one, and usually 5-15, ES cells are introduced into the cloned embryo either by aggregation techniques or blastocyst injection according to methods known by those of moderate skill in the art. These ‘secondary’ ES cells are introduced intact and may either be derived from the same culture as the one from which the nucleus donor came, or a continuation of that culture, or a different culture, or a mixture. One function of the secondary ES cells is to rescue or enhance the developmental potential of the cloned embryo, such that it has a greater probability of developing fully. The resulting embryo now contains a mixture of cells from the clonally derived embryo and secondarily introduced ES cells. The mixed cell embryo is then transferred into a female surrogate recipient, wherein the embryo develops into a viable fetus. Where the same ES cell culture is used both the nucleus donor and the secondary ES cells the resulting embryo is not genetically chimaeric. Where a different ES cell culture is used, the resulting embryo may be genetically chimaeric.

In another embodiment of the invention, cells reconstituted following the transfer of nuclear components to an enucleated oocyte are subjected to a signal to activate embryonic development in vitro, and cultured as described. However, the resultant embryos are used to derive cell lines by further culture in vitro. In a preferred embodiment, embryos are cultured to the blastocyst stage and used to derive embryonic stem (ES) cell lines or ES cell-like lines, according to methods known by those skilled in the art. In a further embodiment, cells of the lines derived in this way are induced to differentiate along prescribed pathways by varying in vitro culture conditions. ES or ES cell-like cells can be induced by those skilled in the art to differentiate to produce populations of a variety of cell types, including without limitation, cardiomyocytes (Klug, et al., J. Clin. Invest. 98, 216 [1996]), neuronal cells (Bain, et al., Dev. Biol. 168, 342 [1995]) or blood cells (Wiles, & Keller, Development 111, 259 [1991]). Such cells have great utility, as for example in the emergent field of tissue engineering (described in: Kaihara & Vacanti, Arch. Surg. 134, 1184 [1999]).

Microinjection has many advantages, relating to the delivery of an ES cell nucleus into an enucleated oocyte and the resultant reconstitution of the ES cell nucleus, including the following. First, total or partial nucleus delivery (i.e., partial delivery into an enucleated oocyte and the resultant reconstitution of the ES nucleus that encompasses nuclear constituents including chromosomal constituents) by microinjection is applicable to a wide variety of cell types—whether grown in vitro or in vivo—irrespective of size, morphology, developmental stage of nuclear donor, and the like. Second, nucleus delivery by microinjection enables careful control of the volume of nucleus donor cell cytoplasm and nucleoplasm co-introduced into the enucleated oocyte at the time of nuclear injection. This is particularly germane where extraneous material adversely affects developmental potential. Third, nucleus delivery by microinjection allows carefully controlled co-injection (with the donor nucleus) of additional agents into the oocyte at the time of nuclear injection: these agents are exemplified below. Fourth, nucleus delivery by microinjection readily allows a period of exposure of the donor nucleus to the cytoplasm of the enucleated oocyte prior to activation. This exposure may facilitate chromatin remodeling, reprogramming or other changes in the transferred chromatin (such as the recruitment of maternally-derived transcription factors) which favor subsequent embryonic development. Fifth, nucleus delivery by microinjection allows a wide range of choices of subsequent activation protocol (in one embodiment, the use of Sr²⁺); different activation protocols may exert different effects on developmental potential. Sixth, activation may be in the presence of microfilament-disrupting agents (in one embodiment, cytochalasin B) to prevent chromosome extrusion, and modifiers of cellular differentiation (in different embodiments, dimethylsulfoxide, or 9-cis-retinoic acid) to promote favorable developmental outcome. Seventh, in one embodiment, nucleus delivery is by piezo electrically-actuated microinjection, allowing rapid and efficient processing of samples and thereby reducing trauma to cells undergoing manipulation. This trauma reduction is, in part, because donor cell nucleus preparation and introduction into the enucleated oocyte may be performed with the same injection needle; contrastingly, the employment of conventional microinjection needles would require at least one change of needle between coring of the zona pellucida and puncturing of the oocyte plasma membrane. Eighth, not only individual steps, but their inter-relationship, is a feature of the method of the invention. We now present those individual steps in greater detail and show how they are arranged in respect of one to the other in the present invention.

Detailed description 1: The recipient oocyte. The stage of oocyte maturation in vivo prior to harvesting for enucleation and in preparation as a recipient for nuclear transfer potentially influences the outcome of cloning methods. Injection of the donor nucleus may be into oocytes or their progenitors at any stage of development. A preferred embodiment of the invention transfers nuclei into mature, mII oocytes as recipients; such mII oocytes are of the type normally activated by fertilizing spermatozoa. The chemistry of the oocyte cytoplasm changes throughout the maturation process. This is exemplified by Metaphase Promoting Factor (MPF) a dimeric complex of cyclin B2 and cdc2 protein kinase. Cells in which MPF activity is high are at metaphase of the cell cycle. For example, in the mouse, the cytoplasmic activities associated with MPF are maximal in those immature oocytes which are arrested at Metaphase of the first meiotic division (metaphase I, mI). MPF activity then declines with the extrusion of the first polar body (Pb1), again reaching high levels at the second metaphase, mII. These high levels are sustained and serve to arrest oocytes at mII, rapidly diminishing when the oocyte receives a signal to resume the cell cycle (activation), such as the signal delivered by a fertilizing sperm or Sr²⁺. Where an ES cell nucleus is injected into the cytoplasm of a mII oocyte, the high MPF activity causes the break-down of its nuclear envelope, with attendant chromatin condensation, resulting in the formation of ES cell-derived metaphase chromosomes.

Oocytes that may be used in the method of the invention include both immature stage oocytes (such as those with an intact nucleus, known as a germinal vesicle) and mature stage oocytes (that is, those at mII). Mature oocytes may be obtained, for example, by inducing an animal to super-ovulate by injecting gonandotrophic or other hormones (for example, sequential administration of equine and human chorionic gonandotrophins) and surgical harvesting of ova shortly after ovulation (for example, 13-15 hours after the onset of estrous in the mouse, 72-96 hours after the onset of estrous in the cow and 80-84 hours after the onset of estrous in the domestic cat).

Where oocyte availability is restricted to immature oocytes, they may be cultured in a maturation-promoting medium until they have progressed to mII; this is known as in vitro maturation (IVM). Methods for IVM of immature bovine oocytes are described in WO 98/07841, and for immature mouse oocytes in Eppig & Telfer (Mets. Enzymol. [Academic Press] 225, pp. 77-84, [1993]). In a further embodiment of the invention, immature oocytes may be used as recipient cells without IVM, e.g. the oocytes may be matured in vitro prior to enucleation.

Detailed description 2: Oocyte enucleation. Oocyte enucleation may be performed by a method known in the art. Preferably, the oocyte is exposed to a medium containing an inhibitor of microtubule and/or microfilament assembly prior to and during enucleation. Disruption of actin-containing microfilaments or tubulin-containing microtubules imparts relative fluidity to the cell membrane and/or underlying cortical cytoplasm, such that a portion of the oocyte enclosed within a membrane can easily be aspirated into a pipette with minimal damage to subcellular structures. A microfilament-disrupting agent of choice is cytochalasin B (5μ/ml). Suitable microtubule-disrupting agents, such as nocodazole, 6-dimethylaminopurine and colchicine, are also known to those skilled in the art. Additional microfilament disrupting agents include, but are not limited to cytochalasin D, jasplakinolide, latrunculin A, and the like.

In a preferred embodiment of the invention, enucleation of the mII oocyte is achieved by aspiration using a piezo electrically-actuated micropipette. Throughout the enucleation microsurgery, the mII oocyte is anchored by a conventional holding micropipette. The flat tip of a piezo electrically-driven enucleation micropipette (internal diameter≈7 μm) is brought into contact with the zona pellucida. A suitable piezo electric driving unit is sold under the name of Piezo Micromanipulator/Piezo Impact Drive Unit by Prime Tech Ltd. (Tsukuba, Ibaraki-ken, Japan). The unit utilizes the piezo electric effect to advance, in a highly controlled, rapid manner, the microinjection pipette tip a short distance (approximately 0.5 μm). The intensity and interval between each pulse can be varied and regulated by a control unit. Piezo pulses (for example, intensity=1-5, speed=4-16) are applied to advance (or drill) the micropipette through the zona pellucida while maintaining a small negative pressure within it. In this way, the micropipette tip rapidly passes through the zona pellucida and is thus advanced to a position adjacent to the mII plate (which contains the chromosome-spindle complex and is discernible as a translucent region in the cytoplasm of the mII oocytes of several species, often lying near the first polar body). Oocyte cytoplasm containing the metaphase plate is then gently and briskly aspirated into the microinjection pipette in the minimal volume and the injection pipette (now containing the mII chromosomes) withdrawn. The effect of this procedure is to cause a pinching off of that part of the oocyte cytoplasm containing the mII chromosomes. The microinjection pipette is then pulled clear of the zona pellucida and the chromosomes discharged into surrounding medium prior to microsurgical removal of chromosomes from the next oocyte. Where appropriate, batches of oocytes may be screened to confirm complete enucleation. For oocytes with granular cytoplasm (such as porcine, ovine and feline oocytes), staining with a DNA-specific fluorochrome (for example, Hoeschst 33342) and brief examination under low intensity UV illumination (in some cases enhanced by an image intensified video monitor) is advantageous in determining the efficiency of enucleation.

Enucleation of the mII oocyte may be achieved by other methods, such as that described in U.S. Pat. No. 4,994,384. For example, enucleation may be accomplished microsurgically using a conventional micropipette, as opposed to a piezo electrically-driven one. Enucleation can be achieved by first slitting the zona pellucida of the oocyte with a glass needle along 10-20% of its circumference and close to the position of the mII chromosomes. The oocyte is resident in a drop of medium containing cytochalasin B on the microscope stage. Chromosomes are removed with an enucleation pipette having an unsharpened, beveled tip.

After enucleation, oocytes are ready to receive ES cell nuclei. It is preferred to prepare enucleated oocytes within about 2 hours of donor nucleus insertion.

Detailed description 3: Preparation and maintenance of ES cell lines. The isolation, culture and manipulation of ES cells is described, for example, in: Hogan, et al., Manipulating the mouse embryo-2nd ed. (Cold Spring Harbor Laboratory Press) (1994). Elements of this description are herein summarized.

Primary mouse ES cells may be isolated from expanded blastocysts at least approximately 3.5 days post-activation of development (such as fertilization). Embryos are flushed from the uterine horns of animals with a medium such as DMEM (supplemented with 10% fetal calf serum and 25 mM HEPES, pH 7.4) and placed individually into 10 mm well tissue culture dishes containing a preformed layer of feeder cells, described below, and 1 ml of ES cell culture medium. This initial stage of embryo culture may also be performed in small drops of ES medium without feeder cells incubated under light paraffin oil. After 1-2 days of further culture, the embryos ‘hatch’ from the zona pellucida and attach to the surface of the tissue culture dish by migration of cells of the trophectodermal (TE) lineage. Shortly after embryo attachment the inner cell mass (ICM) becomes readily distinguishable from cells of the TE lineage (trophoblasts) and grow rapidly. After a total of 4-5 days of blastocyst culture, (ES) cells derived from the ICM are dislodged from the underlying cells using the sealed end of a finely drawn pasteur pipette.

Cells are treated with trypsin to disaggregate the ES cell clump into smaller groups usually containing of 3 or 4 cells. These are then transferred to a fresh feeder cell tissue culture well. Primary ES cell-like colonies are identifiable by their morphology, as described below.

ES cells and their genetically engineered derivatives are cultured under stringent growth conditions in order that they retain a normal karyotype; this is necessary to ensure that they have the potential to contribute at a working frequency to functional germ cells. It is known that suboptimal culture conditions may give rise to ES cell variants that have undergone karyotypic changes, chromosomal rearrangements and/or other mutations that increase their growth rate and decrease their ability to differentiate in vivo. Optimal culture conditions are known to those skilled in the art of culturing ES cells and include supplying necessary concentrations of nutrients and growth factors and avoiding culturing cells at very high density. Cells cultured at high density have a propensity to form clumps whose surface cells differentiate into endodermal-like cells with a restricted pluripotency. Favorable culture densities may be achieved by splitting the cultures 1:2 to 1:6 every 2-3 days and causing small groups of 3-4 cells to dissociate further into single cells after mild treatment with the protease, trypsin, according to standard methods. Healthy ES cells in culture typically grow in tightly packed groups with ‘smooth’ outlines. The presence on colony surfaces of ‘rough’ endoderm, or the spreading of cells onto the substratum, are amongst indications of suboptimal culture conditions known to those of moderate skill in the art.

All culture medium, supplements, and the like, are endotoxin-free. The culture medium most frequently used is Dulbecco's modified Eagle's medium (DMEM) and 4.5 mg/ml glucose, with optional 1 mM sodium pyruvate. DMEM is a bicarbonate-buffered culture medium designed to give a pH of 7.2-7.4 in an atmosphere of 5% CO₂ in air at approximately 35° C. DMEM is usually be supplemented just before use with: (a) 2 mM glutamine; 0.1 mM nonessential amino acids; (c) 0.1 mM β-mercaptoethanol; (d) 50 μg/ml gentamycin, or 100 U/ml each penicillin and streptomycin, or no antibiotics; (e) 15% fetal calf serum (FCS; see below); and optionally, (f) leukemia inhibitory factor (LIF), also known as differentiation inhibitory factor (DIA) (see below).

For subculture and harvesting of the ES cells, they are detached from tissue culture dishes and dissociated from one another by treatment with a mixture of trypsin and disodium ethlenediamine tetraacetic acid (EDTA) (for example, at final concentrations of 0.025% and 75 mM, respectively) in Ca²⁺—Mg²⁺-free phosphate-buffered saline.

FCS, also known as fetal bovine serum, is used to supplement the DMEM for ES cell culture. Typically the FCS is used at 15% (v/v). However, lower concentrations (for example, 1-5%) of FCS support culture of ES cells whose nuclei are competent to direct the development of fetuses and live offspring in the method of the invention. Moreover, these lower concentrations of FCS support an actively growing culture, implying that cells at all stages of the cell cycle may be represented therein, and which may be employed in the method of the invention.

Leukemia inhibitory factor (LIF) is a secretory cytokine that inhibits the spontaneous differentiation of ES cells. It is one of the active components of Buffalo-rat-liver (BRL) cell conditioned medium that is known to be used to grow ES cells. In ES cell co-culture, feeder cells express LIF in an active form, although the medium may be supplemented with purified LIF. Cell-free medium conditioned by feeder cells is not sufficient to support ES cell culture, requiring that it is supplemented with, for example, purified LIF (see below).

Although it is possible to culture ES cells in the absence of feeder cells in medium supplemented with LIF, most laboratories rely on a feeder layer to provide factors that enhance the proliferation of and maintain the undifferentiated state of ES cells. The two kinds of feeder cells most commonly used are primary cultures of mouse embryo fibroblasts (MEFs), harvested from 12.5 to 14.5 dpc embryos by methods known to those skilled in the art, and the STO mouse fibroblast cell line which is a thioguanine- and ouabain-resistant subline of SIM mouse fibroblasts. Mitotically inactive feeder cells are prepared by treatment with mitomycin C or by γ-irradiation.

Methods of deriving ES cell-like cells have been described for other species, including cattle (Cibelli, et al., Theriogenology 47, 241 [1997]), hamster, (Doetschman, et al., Dev. Biol. 127, 224 [1988]), human (Thomson, et al., Science 282, 1145 [1998]) and rabbit (Schoonjans et al., Mol. Reprod. Dev. 45, 439 [1996]). These methods can be applied by one skilled in the art to any appropriate species to derive ES cell-like cells.

Detailed description 4: Preparation of genetically-modified or gene-targeted ES cells. ES cells may be genetically modified by methods known to the art. ES cells are preferably modified by ‘gene targeting’. Gene targeting describes a process whereby a genomic mutation is introduced in a directed, non-random manner. In this way, specific mutations may be introduced within the context of an entire genome. Since ES cells can be used to generate individuals, ES cells containing a gene targeted alteration enable the production of whole animals containing the targeted mutation. An important feature of the method—the design and construction of a ‘targeting construct’—is known to those of moderate skill in the art. Targeting constructs typically contain at least one nucleotide sequence that is not native to the host genome. Non-native sequences correspond to the mutation to be introduced, and are flanked by extensive regions (typically ≧5 kbp) that by contrast are highly conserved with, if not identical to, those of the host genome. This means that once inside the cell, the conserved/identical sequences are able to undergo homologous recombination with their complementary counterparts resident upon the target genome.

In order to introduce the mutation into the genome of a given ES cell type, targeting construct DNA is prepared in a relatively pure form and ES cells caused to take up the DNA by a method from a list including infection with wild-type or recombinant retroviruses, lipofection, transfection, and the like, and preferably by electroporation (Hogan, et al., Manipulating the mouse embryo. 2nd ed. [Cold Spring Harbor Laboratory Press], pp. 277-278 [1994]; Joyner [ed], Gene targeting. [Oxford University Press] [1993]).

The efficiency of gene targeting depends on combinations of variables which may be unique to each targeting construct sequence, DNA preparation or ES cell line; however, these merely require routine experimentation within the skill of the art. For example, efficiencies may be affected by the use of isogenic versus non-isogenic DNA, the length of complementary sequence within the targeting construct, the extent of continuous stretches of sequence identity between the targeting DNA and the endogenous gene, the length of complementarity on each flank of the targeting DNA, and the like. Methods for producing gene-targeted ES cells are well known to those skilled in the art. Exemplary gene-targeted ES cells suitable for use in the invention include, but are not limited to, those described in: Mombaerts, et al., Proc. Nad. Acad. Sci. USA, 88, 3084 (1991); Mombaerts, et al., Nature 360, 225 (1992); Itohara, et al., Cell 72, 337 (1993); U.S. Pat. No. 5,859,307, and the like.

Detailed description 5: Preparation of ES cell donor nuclei. Following culture, non-confluent cultures of ES cells are detached from tissue culture dishes and dissociated from one another by treatment with a mixture of trypsin and ethylenediamine tetraacetic acid (EDTA) (for example, in a final concentration of 0.025% and 75 mM respectively), in Ca²⁺- and Mg²⁺-free phosphate-buffered saline. Cell suspensions are then transferred to a drop of CZB•H medium containing 12% polyvinylpyrrolidone on the microscope stage.

Detailed description 6: Insertion of the donor nucleus into the enucleated oocyte. Nuclei (or nuclear constituents including at least the chromosomes) may be injected directly into the cytoplasm of the enucleated oocyte by a microinjection technique. In a preferred method of injection of nuclei from ES cells into enucleated oocytes, a piezo electrically-driven micropipette is used in which one may essentially use the equipment and techniques described above (with respect to enucleation of oocytes) with modifications here detailed.

For example, a microinjection needle is prepared as previously described, such that it has a flush tip with an inner diameter of about 5 μm. The needle may contain mercury near its tip and it is housed in a piezo electrically-actuated unit according to the instructions of the vendor. The presence of a mercury droplet near the tip of the microinjection pipette increases the momentum inherent to the tip advancement and therefore augments tip penetrating capability in a controlled manner. The tip of a microinjection pipette containing individually selected nuclei is brought into intimate contact with the zona pellucida of an enucleated oocyte and several piezo pulses (applied with adjustment using controller setting scales which may be of intensity 1-5, speed 4-6) are applied to advance the micropipette whilst optionally maintaining a light negative pressure within. When the pipette tip has passed through the zona pellucida, the resultant zona ‘core’ is expelled into the perivitelline space and the preselected nucleus within the micropipette is advanced until near the tip. The pipette tip is then apposed to the plasma membrane (oolemma) and advanced (toward the opposite face of the oocyte) until almost at the opposite side of the oocyte cortex. The oocyte plasma membrane is now deeply invaginated around the lip of the injection needle. Upon application of one to two piezo pulses (for example, intensity 1-2, speed 1), the plasma membrane is punctured at the tip as indicated by a rapid—and typically discernible—relaxation of the oolemma. The nucleus is then expelled into the ooplasm with a minimum amount (≦˜1 pl) of accompanying medium. The micropipette is then carefully withdrawn, leaving the newly introduced nucleus within the cytoplasm of the oocyte. The method is performed briskly, typically in batches of 15-20 enucleated oocytes, which at all other times are maintained in culture conditions.

Alternative variants may be used to insert the donor nucleus by conventional microinjection. A description of one such method employing conventional microinjection to insert sperm nuclei into hamster oocytes, is described in: Yanagida, Biol. Reprod. 44, 440 (1991), the disclosure of which pertaining to such method is hereby incorporated by reference.

Detailed description 7: Co-insertion with the donor nucleus of development-modulatory factors. In one embodiment of the invention, one or more agents with the potential to alter the embryo developmental outcome may be introduced prior to, during, or after the combining of the donor nucleus with the enucleated oocyte. For example, nuclei may be co-injected with function-modulating antibodies directed against proteins with hypothetical or known potential to influence the outcome of the method of the invention. Such molecules may include, but are not limited to, proteins involved in vesicle transport (e.g., synaptotagmins), those which may mediate chromatin-ooplasm communication (e.g., DNA damage cell cycle check-point molecules such as Chk1), those with a putative role in oocyte signaling (e.g., the transcription factor, STAT3) or those which modify DNA (e.g., DNA methyltransferases). Members of these classes of molecules may also be the (indirect) targets of modulatory pharmacological agents introduced by microinjection in the method of the invention, and which have function-modulating roles analogous to those of antibodies. Both antibodies and pharmacological agents work by binding to their respective target molecules or the ligands of their respective target molecules. Where the target has inhibitory effect on development outcome, this binding reduces target function, and where the target has a positive effect on developmental outcome, the binding promotes that function. Alternatively, modulation of functions important in the cloning process may be achieved directly by the injection these factors (or factors with analogous activities) rather than agents which bind to them.

In a further embodiment of the invention, ribonucleic acid (RNA) or deoxyribonucleic acid (DNA) may be introduced into the oocyte by microinjection prior to or following donor nucleus insertion. For example, injection of recombinant DNA harboring the necessary cis-active signals may result in the transcription of sequences present on the recombinant DNA by resident or co-injected transcription factors; subsequent expression of encoded proteins would either have an antagonistic effect on factors inhibitory to embryo development or an enhancing effect on positive ones. Moreover, the transcript may possess antisense regulatory activity towards mRNAs encoding proteins that diminish developmental potential. Alternatively, such regulation may be achieved by direct delivery of nucleic acids (or their derivatives) with an antisense function (e.g., antisense mRNA); this obviates the need for transcription within the oocyte to produce the antisense regulatory molecule. In a favored embodiment, this delivery is by microinjection. Finally, the transcript may exert a critical influence on the transcriptional regulation of gene expression in the early embryo. Such an influence could also be mediated by the microinjection of additional molecular species able to affect translation.

Recombinant DNA (either circular or linear) introduced by the method of the invention may comprise a functional replicon containing one or more expressed, functional genes. The genes may be under the control of one or more promoters whose activities may exhibit a narrow, broad or intermediate developmental expression profile. For example, a promoter active exclusively in the early zygote would direct immediate, but brief expression of its associated gene. Introduced DNA may be lost during embryonic development or integrate at one or more genomic loci, to be stably replicated throughout the life of the resulting transgenic individual. In one embodiment, DNA constructs encoding putative ‘anti-aging’ proteins, such as telomerase, superoxide dismutase or other oxidation-protective proteins, may be introduced into the oocyte by microinjection. Alternatively, proteins may be injected directly therein, such as sperm factor proteins.

Detailed description 8: Activation of development of the reconstituted cell. In one embodiment of the invention, enucleated oocytes that had received a donor nucleus, are returned to culture conditions for 0-6 hours prior to activation; thus, oocytes may be activated at any time up to approximately 6 hours after insertion of the donor nucleus into the enucleated oocyte. We here refer to this interval as the ‘latent period’. In a preferred embodiment, the latent period is 1-3 hours. Activation may be, without limitation, electrically, by injection of one or more oocyte-activating substances, or by transfer of the oocytes into media containing one or more oocyte-activating substances.

Reagents capable of providing an activating stimulus (or combination of activating stimuli) include, but are not limited to, cytosolic factors from sperm (exemplified by the protein responsible for the soluble activity, oscillogen) and certain pharmacological compounds (exemplified by 6-dimethylaminopurine [DMAP], IP₃ and other signal transduction modulators); these may be introduced by microinjection prior to, concomitantly with, or following reconstitution of the cell by donor nucleus insertion. One or more activating stimuli may be provided following transfer of reconstituted cells (either immediately or following a latent period) to media containing one or members of a sub-set of activating compounds. This sub-set includes without limitation, stimulators of Ca²⁺ release (e.g., caffeine, ethanol, and Ca²⁺ ionophores such as A23187 and ionomycin), modulators of phosphoprotein signaling (e.g., 2-aminopurine, staurosporine and sphingosine), inhibitors of protein synthesis (e.g., A23187 and cyclohexamide), DMAP, or combinations of the foregoing (e.g., DMAP plus ionomycin). In one embodiment of the invention, activation of reconstituted cells is achieved by culture for 1-6 hours in Ca²⁺-free CZB medium containing divalent strontium ions, Sr²⁺, furnished in 10 mM SrCl₂.

In embodiments of the invention wherein the activating stimulus is applied concurrently with or after donor nucleus insertion, reconstituted cells may be transferred to a medium containing one or more microfilament-disrupting agents such as cytochalasin B at 5 μg/ml in dimethyl sulfoxide on or soon after application of the activating stimulus; this inhibits cytokinesis and hence the loss of chromosomes via a pseudo-polar body. Incubation in the presence of a cytokinesis inhibitor is for a period of 4-12 hours, but more preferably, 6 hours. This embodiment is preferably applied where the donor nucleus contains 2C DNA.

In another embodiment of the invention, enucleated oocytes may be activated prior to donor nucleus insertion, by activation methods described above. Following exposure to an activating stimulus, oocytes may be cultured for up to approximately 6 hours prior to injection of a 2C nucleus as described above. In this embodiment, newly-introduced chromosomes rapidly become associated with pronucleus-like structures and it is not desirable to suppress pseudo-polar body extrusion by culture with a cytokinesis-preventing agent.

Detailed description 9: Development to produce viable fetuses and offspring. The reconstituted cell is activated to produce a pronuclear, 1-cell embryo that may be allowed to develop by culture in vitro. Where pseudo-polar body extrusion was suppressed by exposure of the embryo to cytokinesis blocking agents, the embryo is transferred to fresh medium lacking microfilament-, or microtubule-disrupting agents. Culture may continue to the 2-cell to morula/blastocyst stages, at which time the embryo may be transferred into the oviduct or uterus of a pseudo-pregnant surrogate mother.

Alternatively, the embryo may be split and the cells clonally expanded, for the purpose of improving yield by augmenting the number of offspring derived from a single cell reconstitution.

In a further embodiment, embryos derived by the method of the invention are used to generate further embryos by serial nuclear transfer. To achieve this, reconstituted cells are activated and allowed to develop by in vitro culture as described above. In another embodiment, the culture may be in vivo following transfer to a suitable surrogate mother. Following continued culture for several days, preferably 3-5 days, cells from the resulting embryos are dispersed by mild treatment with a protease such as trypsin, or by mechanical methods known by those skilled in the art. Individual cells from these embryos are then used as nucleus donors; the nucleus of each may be removed and inserted into an enucleated oocyte, which is subsequently activated and allowed to undergo development. The methods of donor nucleus insertion, enucleation, activation of development and embryo culture are described above.

Detailed description 10: Production of populations of differentiated cells. In an additional embodiment, cloned embryos generated by the method of the invention are used to establish ES cell-like cell cultures in vitro. This is achieved by methods known to those skilled in the art and described in: Hogan, et al., Manipulating the mouse embryo. 2nd ed. (Cold Spring Harbor Laboratory Press), 265-272 (1994). Such cultures may be induced to undergo differentiation in a prescribed manner, thereby generating potentially unlimited sources of enriched cells of a particular genotype. Methods of inducing such differentiation have been described to obtain enriched populations of neuronal cells (Bain, et al., Dev. Biol. 168, 342 [1995]), cardiomyocytes (Klug, et al., J. Clin. Invest. 98, 216 [1996]) and hematopoietic cells (Wiles & Keller, Development 111, 259 [1991]. As an example, this allows the amplification of immunologically matched cells for use in transplantation. The cells may be thus matched because they are clonally derived by the method of the invention from the transplant recipient. In another embodiment, the amplified cells are genetically modified, for example, such that they no longer express molecular targets of immune surveillance, such as the Galα1-3Gal moiety which prevents the successful transplantation of non-primate-derived cells into primates. The growth of clonally-derived cells on matrices in vitro provides a link between the technologies of cloning and tissue engineering (Kaihara & Vacanti, Arch. Surg. 134, 1184 [1999]). Populations of cells produced by the method of the invention therefore have utility in transplant medicine.

### DEFINITIONS USED HEREIN

2C, 4C: The genomic complement of the cell. 1 C represents the unit genome, thereby defining “C”. 1 C represents the genome of a haploid, prereplicative cell, in which each locus is represented once.

2n: The diploid state of a cell, with “n” referring to the haploid (unit) number of chromosomes.

Differentiate: Process by which a cell population becomes increasingly specialized, usually as a result of changes in gene expression.

Cloned animal: Animal produced by cloning. Non-chimaeric metazoan whose nuclear genome is derived from a single cell.

Cloning: The production of populations of differentiated cells following the transfer of nuclear chromosomes from a nucleus donor cell to a recipient cell from which the resident chromosomes had been removed; the method preferably utilizes an enucleated oocyte as the recipient cell. This can result in the development of offspring whose non-mitochondrial DNA is derived from a single cultured cell, the nucleus donor.

Egg: An oocyte or recently fertilized female gamete.

Embryo: Any stage subsequent to the developmental activation of an oocyte, or any stage subsequent to a step that mimics activation of an oocyte in another cell type.

Embryonic stem (ES) cells: Those derived from the inner cell mass (ICM) of preimplantation embryos (blastocysts) with the following properties: (i) they are amenable to long-term laboratory culture and storage, (ii) they retain their undifferentiated state, (iii) they retain their 2n ploidy, (iv) they are able to resume their developmental program and differentiate into any cell type, including functional germ cells, if mixed with the cells of a embryo and cultured to form a chimaeric embryo. ES cells exhibit homologous recombination that can be manipulated, as in gene targeting.

ES cell-like cells: Cultured cells derived from the ICM of blastocysts, but for which ES cell properties have not been completely demonstrated.

Fetus: Stage of development after placentation and prior to term (birth or delivery of offspring).

Live-born: Living offspring.

Microfilament: Cytoskeletal polymeric actin.

Microtubule: Sub-cellular filaments comprised of tubulin subunits that anchor and orientate chromosomes.

Nucleus: The entire nucleus or a portion thereof, wherein the nuclear contents include at least the minimum material able to direct development in a cell lacking any other non-mitochondrial genome.

Offspring: Individual developing at least to term.

Oocyte: Female gamete that has undergone the first metaphase in meiosis and is arrested at the second (metaphase II). Oocytes are therefore not fertilized but are at the developmental stage that participates in normal fertilization. Oocytes may be generated in vivo following ovulation, or may be the result of maturation of immature, surgically isolated precursors that are subsequently allowed to mature in vitro.

Pluripotent: The capacity to differentiate into any one of a multiplicity of cell types. It typically describes stem cells.

Reconstituted cell: A cell made by the process of inserting into an enucleated cell additional materials which include at least the minimal complement of chromosomes present in a nucleus donor cell necessary to direct sustained development. In a preferred embodiment, a reconstituted cell is an enucleated oocyte that has had the nucleus of an ES cell inserted into it.

Term: Full-term. Having undergone the full program of embryonic development in utero, corresponding to the gestation period.

Zygote: A recently-fertilized female gamete, also known as a 1-cell embryo.

### EXAMPLES

The following examples illustrate the method of the invention and the development of live offspring from oocytes injected with nuclei of cells from the ES cell lines E.14, AB2.2 and R1. These represent well-established and widely available cell lines originally derived from F1 and inbred strains of mice. M72 is a derivative of E.14 carrying a targeted mutation. The following examples are intended to serve as illustrative examples of animal oocytes, ES cells, ES cell-like cells, media and applications that may be used in the process of the invention, and are not intended to be limiting; further examples of embodiments of the invention would readily be recognized by those skilled in the art.

Reagents. All organic and inorganic compounds are laboratory grade or higher and were purchased from Sigma Chemical Co. (St. Louis, Mo.) unless stated otherwise. In general and unless stated otherwise, oocyte culture was in CZB medium (Chatot, et al., 1989. J. Reprod Fert. 86, 679-688) supplemented with 5.56 mM D-glucose. CZB medium is: 81.6 mM NaCl, 4.8 mM KCl, 1.7 mM CaCl₂, 1.2 mM MgSO₄, 1.8 mM KH₂PO₄, 25.1 mM NaHCO₃, 0.1 mM Na₂EDTA, 31 mM Na.lactate, 0.3 mM Na.pyuvate, 7 U/ml penicillin G, 5 U/ml streptomycin sulfate, and 4 mg/ml bovine serum albumin (BSA). Collection of oviductal, ovulated oocytes and their subsequent micromanipulation on the microscope stage was in a modified CZB (herein termed CZB•H) which is CZB supplemented with 20 mM Hepes but with reduced concentrations of NaHCO₃ (5 mM) and BSA (3 mg/ml); CZB•H has a pH of 7.4. BSA in CZB•H may be replaced with 0.1 mg/ml polyvinyl alcohol (PVA; cold water soluble, average relative molecular mass=103); the function of both BSA and PVA is to reduce stickiness the wall of the injection pipette during micromanipulation. The lubricant effect of PVA lasts longer than that of BSA making its inclusion desirable during repeated use of a single micropipette for extensive micromanipulation. Where appropriate, oocytes or reconstituted cells were cultured in CZB lacking CaCl₂ (i.e., Ca²⁺) but supplemented with agents to induce oocyte activation and, in some cases, suppress cytokinesis.

ES cells were cultured in Dulbecco's Modified Eagle's Medium (DMEM) for ES cells (Specialty Media, Lavallette, N.J.), supplemented with 0.5%-15% (v/v) heated-inactivated fetal calf serum (FCS; HyClone Laboratories, Logan, Utah), 100 U/ml penicillin-100 μg/ml streptomycin (Specialty Media), 0.2 mM L,-glutamic acid (Specialty Media), 1% (v/v) non-essential amino acid cocktail (Specialty Media), 1% (v/v) 2-β-mercaptoethanol (Specialty Media), 1% (v/v) nucleoside cocktail (Specialty Media), and 1000 U/ml recombinant leukemia inhibitory factor (LIF) (GIBCO, Grand Island, N.Y.). FCS was heat-inactivated at 56° C. for 25 min prior to use.

Animals. Animals used in these examples were maintained in accordance with Federal guidelines prepared by the Committee on Care and Use of Laboratory Animals for the Institute of Laboratory Resources National Research Council (DHEW publication no. [NIH] 80-23, revised in 1985).

### Example 1

### Preparation of Nuclear Donor Cells from the % Well-Established ES Cell Line, E14

This example utilizes the well-established and widely available ES cell line, E14 as the source of nuclei for microinjection into enucleated mouse oocytes. The E14 cell line was derived from strain 129/Ola mouse blastocysts (Hooper, et al., Nature 326, 292 [1987]). The 129/Ola parent strain is homozygous for the A (agouti) gene, with a chinchilla coat color that reflects its c^{ch}p/c^{ch}p genotype (chinchilla coat coloring is a soft-yellow). The ES cell line, E14, was derived from one such mouse strain; 129/Ola, in the laboratory of Dr. Martin Hooper in Edinburgh, UK. To recognize offspring cloned from ES cell nuclei by the coat color of said offspring, it is necessary to select oocyte donor and foster mother strains whose coat colors differ from that of the mouse strain from which the ES cell is derived. In one embodiment, the nuclei of E14 cells (genetically chinchilla) are transferred into enucleated B6D2F1 oocytes (genetically black) and reconstituted cells allowed to develop following transfer into CD-1 surrogate mothers (genetically white).

A low passage aliquot of E14 cells (ie one that had been passaged fewer than 11 times) was obtained in 1990 and further cultured in three different laboratories, giving a total of 31-39 passages. The choice of E14 cells in the examples reported here was supported by their considerable utility in the generation of gene-targeted mice (Mombaeris, et al., Proc. Nad. Acad. Sci. USA, 88, 3084 [1991]; Mombaerts, et al., Nature 360, 225 [1992]; Itohara, et al., Cell 72, 337 [1993]; Rodriguez, et al., Cell 87, 199 [1999]). Thus, the E14 cells are of proven efficacy in the generation of germ line chimaeras from which strains of gene-targeted mice have been established. The E14 cultures typically exhibited a range of cell diameters from about 10 μm to about 18 μm. Without being bound by theory, it was reasoned that small cells (about 10 μm to about 12 μm) would likely be pre-5-phase and therefore contain a 2C genomic complement (in 2n chromosomes), and that the larger cells (about 16 μm to about 18 μm) were generally post-5-phase, likely containing 2-4C DNA (2n chromosomes).

ES cells were grown in ‘DMEM for ES cells’ (Specialty Media, Phillipsburg, N.J.) supplemented with 0.5-15% (v/v) heat-inactivated fetal calf serum (FCS) (Hyclone), 1000 U leukemia inhibitory factor (LIF)/ml (Gibco), and the following reagents (Specialty Media): 1% (v/v) penicillin-streptomycin, 1% (v/v) L-glutamine, 1% (v/v) non-essential amino acids, 1% (v/v) nucleosides, and 1% (v/v) β-mercaptoethanol. Cells were split 1:3 or 1:4 every 24 hours, reflecting an approximate cell cycle period of 12 hours. Where appropriate, culture was on a feeder layer of mitomycin-C treated primary embryonic fibroblasts derived from embryonic day 13.5 mice. In these cases, ES cells were cultured in feeder-free conditions for at least one week prior to micromanipulation; by the time of nuclear transfer no feeder cells were detectable in the culture.

ES cell culture in the absence of feeder cells was in medium supplemented with 15% (v/v) FCS and 1000 U/ml LIF. Where growth in low [FCS] was desirable, the FCS concentration was reduced stepwise. At a concentration of 5% (v/v) FCS, cells divided almost as vigorously as they did at 15% (v/v), with little overt differentiation. However, growth of the cells slowed noticeably when the FCS concentration was 4% (v/v) or less. Extensive cell death occurred when the cells were cultured in medium with 0.75% or 0.5% (v/v) FCS, conditions which may ‘starve’ certain cell types and cause them to exit the cell cycle (i.e., enter G0).

To prepare suspensions of individual ES cells from cultures, cells were first washed with phosphate-buffered saline (PBS). Detachment of cells from each other and culture vessel was by subsequent treatment with a mixture of trypsin (0.025% [w/v]) and ethylenediaminetetraacetic acid, disodium salt (EDTA; 0.75 mM) in Ca²⁺/Mg²⁺-free PBS. The cells were then washed three times by gentle centrifugation (2000 g for 5 min) and resuspension (twice in DMEM and once in PBS) and resuspended in PBS medium at a concentration of approximately 10⁷/ml.

Up to 2 days prior to ES cell nucleus collection (but usually immediately prior to collection) a drop of approximately 2 μl of the ES cell suspension was mixed with 20 μl of CZB•H supplemented with 12% (w/v) polyvinylpyrrolidone (PVP) (average relative molecular mass, 3.6×10⁵); we here refer to this as CZB•H-PVP. The mixture was transferred to the microscope stage for micromanipulation.

Enucleation of oocytes. Oocyte enucleation was by aspiration into a micropipette (internal diameter 6 μm) that had been advanced through the oocyte zona pellucida by piezo-actuation using Model MB-U unit (Prime Tech Ltd., Tsukuba, Ibaraki-ken, Japan). This unit uses the piezo electric effect to advance the micropipette tip a very short distance (approximately 0.5 μm) per pulse at high speed. The intensity and speed of the pulse were regulated by the controller, with settings typically at 2 and 4 respectively for zona penetration.

Mature oocytes were collected from the oviducts of female, 8-12-week-old B6D2F1 mice caused to superovulate by the serial intraperitoneal administration of 5 U pregnant mare's serum gonadotrophin (PMSG) and 5 U human chorionic gonadotrophin (hCG) respectively 64 and 13-16 hours prior to oocyte collection. Oocytes were freed from surrounding cumulus cells by immediate treatment in CZB•H containing 0.1% (w/v) bovine testicular hyaluronidase (300 U/mg, ICN Biochemicals Inc., Costa Mesa, Calif.) for 5-10 min at 25-30° C. Cumulus-free oocytes were washed four times in CZB•H (lacking hyaluronidase) by serial transfer using a pipette. Washed oocytes were subsequently held in a drop of CZB (10-30 μl) under mineral oil (E.R. Squibb and Sons, Princeton, N.J.) equilibrated in water-saturated, 4% (v/v in air) CO₂ at 37° C. in preparation for micromanipulation.

Groups of cumulus-free oocytes (usually 15-20) were transferred into a droplet of CZB•H containing 5 μg/ml cytochalasin B on the microscope stage. Oocytes undergoing microsurgery were held with a holing pipette and the zona pellucida ‘cored’ following the application of several piezo-pulses to an enucleation pipette. The mII chromosome-spindle complex (identifiable as a translucent region) was aspirated into the pipette with a minimal volume of oocyte cytoplasm. Relatively high temperatures (approaching 30° C.) render mII plates more readily discernable due to their increased translucence. Following enucleation of all oocytes in one group (taking approximately 10 min), they were transferred into cytochalasin B-free CZB and held there for up to 2 hours at 37° C., before their return to the microscope stage for further manipulation.

Transfer of ES cell nuclei into enucleated oocytes by microinjection. Here, ES cell nuclei were transferred into enucleated oocytes prepared as described above. It is favored to perform this transfer with the same micropipette as that used to enucleate the oocytes.

For microinjection of donor nuclei into enucleated oocytes, a microinjection chamber was prepared by employing the cover (approximately 5 mm in depth) of a plastic dish (100 mm×15 mm; Falcon Plastics, Oxnard, Calif., catalogue no. 1001). One or more rows, each consisting of two round droplets and one elongated drop was placed along the center line of the dish. The first droplet (approximately 2 μl; 2 mm diameter), for microinjection pipette washing, was of CZB•H-PVP. The second droplet (approximately 2 μl; 2 mm diameter) contained a suspension of nucleus donor cells in CZB•H-PVP. The third (elongated) droplet (approximately 6 μl; 2×6 mm), for enucleated oocytes, was of CZB•H. The totality of the dish, including the droplets, was submerged in mineral oil (Squibb). The dish was placed on the stage of an inverted microscope equipped with Hoffman Modulation contrast optics, in preparation for micromanipulation.

Microinjection of donor cell nuclei into oocytes was achieved by piezoelectrically actuated microinjection. Nuclei were removed ES donor cells and each subjected to gentle aspiration in and out of the microinjection pipette (approximately 7 μm inner diameter) until their nuclei became largely void of visible cytoplasmic material. This served to free the nuclear constituents of cytoplasmic contaminants. In some cases it was necessary to break the plasma membrane of the donor cell by the application of a small number (typically 1) of piezo pulses (at a low intensity setting). Where breakage of the nuclear membrane occurred non-chromosomal nucleoplasmic components could be washed free.

Each nucleus was microinjected into a separate enucleated oocyte within 5-10 min of its isolation into the pipette. The process of nucleus transfer was usually accelerated by collecting the nuclei of several cells (typically up to 7) to form a line of denuded nuclei within the micropipette, before moving the micropipette into the droplet containing the enucleated oocytes.

An enucleated oocyte was positioned on a microscope stage in a drop of CZB medium containing 5 μg/ml cytochalasin B. The zona pellucida of the enucleated oocyte was apposed to the tip of a holding pipette and fixed in place by the application of gentle suction. The tip of the injection pipette was then advanced towards, and brought into intimate contact with the zona pellucida. Several piezo pulses (e.g., intensity 1-2, speed 1-2) were applied to advance the pipette whilst maintaining a light negative pressure within it. When the tip of the pipette had passed through the zona pellucida, the resultant cylindrical core of zona material within the pipette was expelled into the perivitelline space. The donor nucleus foremost within the injection pipette (which typically contained up to 7 nuclei harvested in rapid succession) was then advanced until it was near to the needle tip. The pipette was, in turn, then caused to advance mechanically until its tip almost reached the opposite side of the oocyte cortex. This created a deep invagination in the enucleated oocyte plasma membrane (oolemma). The invaginated oolemma was then punctured by applying 1 or 2 piezo pulses (typically, intensity 1-2, speed 1) and the ES cell nuclear components expelled into the ooplasm with <1 μl of accompanying medium. The pipette was then gently withdrawn, leaving the nucleus within the ooplasm. Each enucleated oocyte was injected with one nucleus. Approximately 15-20 enucleated oocytes were typically microinjected by this method within 10-15 minutes. All injections were performed at room temperature usually in the range of 25-30° C.

Oocyte activation. ES cell cultures typically contain cells at different stages of the cell cycle, with some containing the 2C complement of DNA typical of 2n cells, and others having undergone a duplicative round of DNA synthesis (S-phase) such that they contain twice this amount (4C DNA) in preparation for cell division. This difference in DNA content is anticipated in the method of the invention, accordingly necessitating different treatments of reconstituted cells following nuclear transfer. Distinction between cells at different stages of the cell cycle (e.g., with different DNA content) is described below; here we correlate cells of relatively small diameter (10-12 μm, referred to as ‘small’) with 2C DNA and those with a relatively large diameter (16-18 μm, referred to as ‘large’) with 4C DNA.

Reconstituted cells corresponding to oocytes that had received nuclei from small ES cells were incubated for 1-3 hours in CZB under mineral oil equilibrated in 4% (v/v) CO₂ in air at saturating humidity at 37° C. These cells were then removed to C²⁺-free CZB containing 10 mM SrCl₂ and 5 μl/ml cytochalasin B (added from a 100× stock in dimethylsulfoxide [DMSO]) for 6 hours. This treatment induced activation of development whilst preventing cytokinesis and, hence, chromosome loss in the form of a pseudo-second polar body. After 6 hours, cells were transferred to fresh CZB medium lacking Sr²⁺/cytochalasin B and incubation continued at 37° C. in 4% (v/v) CO₂ in air at saturating humidity. Hence, normal reductive division after the completion of S-phase was not inhibited after 6 hours.

Reconstituted cells corresponding to oocytes that had received nuclei from large ES cells were incubated for up to 2 hours in CZB under mineral oil equilibrated in 4% (v/v) CO₂ in air at saturating humidity at 37° C. Pre-activation incubation was to allow the synthesis of advantageous macromolecular components (e.g., spindle microtubules) to be functionally completed prior to stimulation of the resumption of meiosis and cytokinesis. Resumption of meiosis (activation) was initiated by transferring cells to Ca²⁺-free CZB containing 10 mM SrCl₂ in 4% (v/v) CO₂ in air at saturating humidity at 37° C., for 1 hour. Note that this medium did not contain cytochalasin B or any other cytokinesis-abrogating agent. Hence, these activated cells underwent extrusion of a pseudo-second polar body. Since the transferred nucleus of the ES donor cell contained 4C DNA, subsequent sister chromatid separation and chromosome loss should have restored embryos to a genomic DNA complement of 2C.

Following activation, reconstituted cells were then transferred to fresh CZB in 4% (v/v) CO₂ in air at saturating humidity at 37° C. for embryo culture. Embryos generated in this way usually possessed 2 pseudo-pronuclei and a single pseudo-second polar body approximately 5 hours post-activation.

Selection of ES nucleus donor cells based on their cell cycle status. We surmised that small cells were in the G1-phase (2C DNA) whilst large cells corresponded to those in G2/M-phases (post S-phase, 4C DNA). This provides a rapid and non-invasive meter of cell ploidy. This assessment is enhanced by the use of ES cell lines engineered to contain a derivative of a non-destructively assayable reporter gene (e.g., the mutant green fluorescent protein, EGFP) under the control of a promoter directing transcription diagnostic of a cell cycle stage. Examples of such promoters include those directing transcription of cyclin D (restricted to G1-phase of the cell cycle) or cyclin B2 (restricted to M-phase of the cell cycle). The reporter protein contains a targeted destruction sequence (destruction box) such as those resident in cyclin proteins. This ensures that its half-life is short, and that its presence reflects promoter activity (and hence the cell cycle stage) rather than longevity of the protein. Where the reporter is EGFP, cells at a given cell cycle stage can be readily and non-invasively identified from within non-synchronous cultures by examination using long-wavelength epifluorescence microscopy; only those cells in which the cell cycle stage-specific promoter is active are fluorescent, allowing their immediate identification and selection as donors for nuclear transfer.

Finally, we exposed R1 ES cells to the microtubule disrupting agent nocodazole (Sigma) at 3 μg/ml for 12 hours. Cultures treated in this way altered dramatically compared to untreated cultures, with the appearance of many rounded and floating cells. Such treatment served to synchronize the ES cell culture by preventing cells from completing metaphase. The genomic content of such cells is 4C, since they have completed a non-reductive round of duplicative DNA synthesis in S-phase.

Embryo transfer. Following 3.5-4 days of culture in a drop of CZB (10-30 μl) under mineral oil (Squibb) equilibrated in water-saturated, 4% (v/v in air) CO₂ at 37° C., morulae/blastocysts were examined and, where appropriate, transferred into the uterine horns of recipient albino CD-1 female mice which had been mated with vasectomized CD-1 males 3 days previously; this establishes appropriate co-ordination between embryonic development and that of the uterine endometrium. Females were either allowed to deliver and raise their surrogate offspring, or else pups were delivered by Caesarian section at 19.5 days post coitum and placed in the care of suitable lactating foster mothers.

### Example 2

### Cloning with ES Cell Nuclei

Experiments were performed in which enucleated oocytes were microinjected with the nuclei of cells from a variety of ES cell lines, exemplifying well-established cell lines originally derived from both inbred and F1 strains of mice. We describe the generation offspring in experiments in which nucleus donor ES cells were cultured in a variety of conditions and further demonstrate the method of the invention with donor cells of different ploidy.

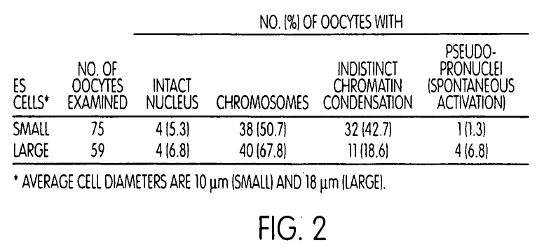

The fate of ES cell chromosomes following nuclear transfer into enucleated oocytes. In experimental Series 1 (FIG. 2), enucleated oocytes received E14 nuclei but were not subjected to an activating stimulus. Such reconstituted oocytes therefore remained in mII. When examined 2-4 hours after microinjection of the nuclei of small cells, 51% of reconstituted oocytes possessed condensed chromosomes arranged in a scattered fashion. By contrast, 68% of oocytes injected with nuclei from large cells possessed condensed chromosomes aligned in a regular array resembling that of maternally-derived chromosomes in mature metaphase II oocytes.

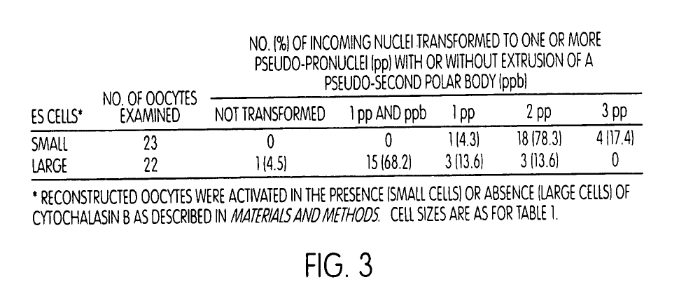

In experimental Series 2 (FIG. 3), we supplied the reconstituted cells with an activation stimulus (strontium ions, Sr²⁺) following nuclear transfer. Anticipating potential differences in the DNA content of small and large cells, we accordingly adapted the nuclear transfer protocol used for each cell type. Oocytes reconstructed with the nucleus of a small cell were removed from CZB culture medium ˜4 hours after nuclear microinjection, and placed into medium containing Sr²⁺ (to activate them) and cytochalasin B (to prevent cytokinesis). We included cytochalasin B because in its absence donor chromosomes would be extruded quasi-randomly into a pseudo-second polar body, generating inviable, hypodiploid embryos. Of the embryos we generated from small cell nuclei, 78% examined ˜6 hours after activation contained two pseudopronuclei (FIG. 3), presumably because the chromosomes within the cell usually formed 2 clusters prior to formation of pseudo-pronuclei.

By contrast, activation of each oocyte reconstructed with the nucleus of a large ES cell was in the absence of cytochalasin B since we reasoned that cytokinetic extrusion of a pseudo-second polar body would be expected to re-establish the normal 2C DNA complement of the reconstituted cell in many such cases. We noted that following activation in the absence of cytochalasin B, 68% of the 1-cell embryos harbored a single pseudo-pronucleus and had emitted a pseudo-second polar body (FIG. 3).

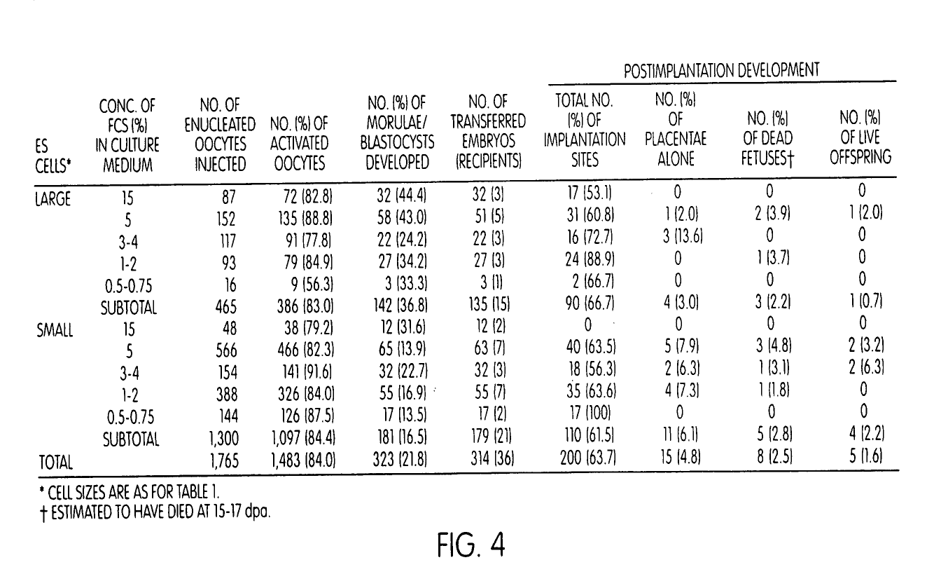

Term development of mice cloned from E14 cells. FIG. 4 summarizes results obtained from experimental Series 3, in which 1765 oocytes were reconstructed using nuclei from E14 cells of different sizes and grown in the presence of different concentrations of FCS. We found no evidence for a marked effect of FCS concentration in the culture medium on the ability of ES cell nuclei to direct development to the morula/blastocyst stage.

Following transfer of the nuclei of small cells, 17% of activated oocytes produced morulae/blastocysts. After transfer into suitable surrogate mothers, 62% of the resultant embryos implanted, giving rise to 9 fetuses at 20 days post activation (dpa); 4 offspring were delivered alive by Cesarean section, and 5 fetuses were developmentally arrested at 15-17 dpa.

One of the live-born pups was euthanized due to lack of a foster mother, and 2 died within 24 h of delivery. One mouse (referred to as ‘Hooper’) survived and is a male with a chinchilla coat color and pink eyes. These characteristics were predicted, because E14 is an XY cell line derived from a male of the 129/Ola mouse strain; 129/Ola mice have a chinchilla coat color and pink eyes. All pups that developed to term were also males with non-pigmented eyes. Hooper has sired three litters with a total of 33 apparently normal pups when crossed with CD-1 females.

Following the transfer of nuclei from large cells, 37% of successfully activated oocytes developed to the morula/blastocyst stage after 3.5 days of culture in vitro. Of the transferred embryos, 67% implanted in the uterus. One full-grown, apparently normal pup and 3 dead fetuses (developmentally arrested at 15-17 dpa) were removed by Cesarean section 20 dpa. We isolated genomic DNA from the placentae of ES cell-derived cloned mice and an ear biopsy from Hooper, and subjected the samples to polymerase chain reaction (PCR) analysis for polymorphic markers and the presence of the Y chromosome-specific gene, Zfy. These analyses further corroborated the E14 provenance of the cloned pups.

The magnitude of these efficiencies means that the method of the invention is readily reproducible. However, the efficiency of the method may be further increased in combination of a supplementary embodiment of the invention in which an embryo is formed from a mixture of ES cells and ES cell-derived embryonic cells generated by nuclear transfer according to the method of the invention.

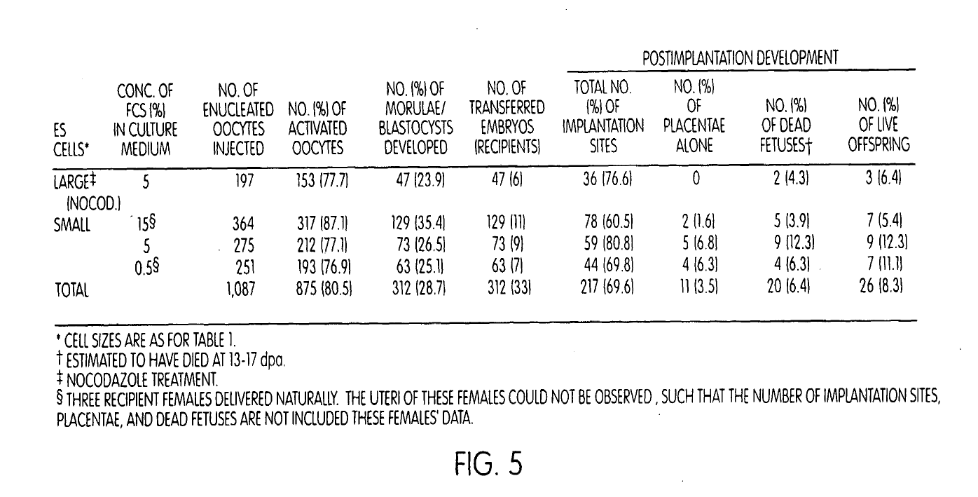

Development of embryos following nuclear transfer from R1 ES Cells. In experimental Series 4 (FIG. 5) we performed 1087 nuclear transfers with the cell line, R1, which is derived from the F1 hybrid, 129/Sv x 129/Sv-CP. There was no pronounced effect of the FCS concentration on cloning outcome. However, the cloning efficiency was markedly higher for R1 cells than for E14 cells. From 314 transferred morulae/blastocysts, 26 live-born cloned pups (8.3%) were obtained. Their clonal provenance is supported by PCR analyses.

Since the nuclei of large E14 cells could, under appropriate experimental conditions, support full development following transfer, in a fifth experimental series (Series 5) we performed analogous experiments with R1 cells. Here, instead of simply selecting large R1 cells, we exposed cultures to nocodazole for 12 hours prior to nuclear transfer, to synchronize the cells in culture at M-phase such that they contained 4C DNA. The proportion of live offspring obtained did not significantly differ from the corresponding value for small R1 cells. Three live-born clones were born. This further suggests that neither nucleus donor ploidy, nor, cell cycle stage are critical parameters in cloning.

### Example 3

### Cloning with the Nuclei of Gene Targeted ES Cells

The utility of the method is illustrated by its use to generate offspring from an ES cell line containing a targeted mutation.

Generation of gene-targeted ES cells. ES cell lines harboring a targeted mutation were derived from E14. This line (described by Zheng & Mombaerts; submitted for publication) was generated by electroporating E14 cells with an M72→VRᵢ2-IRES-tauGFP construct and subsequently cultured as described (Mombaerts, et al., Cell 87, 675 [1996]). One resultant cell line which carried the mutation, T15, yielded chimaeras with extensive colonization of somatic tissues and the germ line following blastocyst injection. We therefore assessed the ability of this line to provide nucleus donors in the method of the cloning invention.

Development of mice cloned from the gene-targeted E14 cell line, T15. Small T15 cells (with an estimated average diameter of approximately 12 μm and ploidy of 2n, 2C) were selected and their nuclei transferred to generate reconstituted cells as described above. 252 cells were successfully reconstructed following T15 nuclear transfer in this way and were cultured in vitro. After 3.5 days of culture, 91 (36%) had developed to the morula/blastocyst stage. These were transferred to pseudo-pregnant foster mothers to enable the continuation of development.

Caesarian section of foster mothers 19.5 days post-coitum revealed 8 dead fetuses (9% of the transferred embryos) and one live-born clone. This shows that nuclei from cells containing targeted mutations can be used clonally to generate offspring by the method of the invention described herein.

### Example 4

### Derivation of ES Cell-Like Cells

Embryos are produced either by in vitro fertilization or by natural mating and recovery. Development of preimplantation embryos to the blastocyst stage in vitro is in G1.2 or G2.2 medium as described by Gardner, et al., Fertil. Steril. 69, 84 (1998). Cells of the ICM of selected blastocysts are immunosurgically isolated using a rabbit antiserum to BeWo cells as previously described (Thomson, et al., Proc. Nad. Acad. Sci. USA 92, 7844 [1995]; Solter, & Knowles, Proc. Nad. Acad. Sci. USA 72, 5099 [1995]). Cells are plated individually into 10 mm well tissue culture dishes containing a preformed layer of irradiated mouse embryonic fibroblasts and 1 ml of culture medium. Culture medium consists of 80% Dulbecco's modified Eagle's medium (no pyruvate, high glucose formulation; Gibco-BRL) supplemented with 20% FCS (Hyclone), 11 mM glutamine, 0.1 mM β-mercaptoethanol (Sigma) and 1% nonessential amino acid stock (GIBCO-BRL).

After 9-15 days of further culture, outgrowths derived from the inner cell mass are dissociated into small clumps typically containing 3 or 4 cells, either by exposure to Ca²⁺- and Mg²⁺-free phosphate-buffered saline containing 1 mM ethylenediamine tetraacetic acid (EDTA), exposure to dispase, or by mechanical dispersal with a pasteur pipette. The smaller clumps are the transferred to a fresh feeder cell tissue culture well. Following further growth, individual colonies with a uniform, undifferentiated morphology were selected and replated as described above.

Primary ES cell-like colonies, identifiable by their morphology, are passaged and expanded by exposure to type IV collagenase (1 mg/ml; GIBCO-BRL) or following selection of individual colonies with a pasteur pipette.

It is known that suboptimal culture conditions may give rise to ES cell variants that have undergone karyotypic changes, chromosomal rearrangements and/or other mutations that increase their growth rate and decrease their ability to differentiate in vivo. Each ES cell-like line is karyotyped at passage 2-7, and those lines with abnormal karyotypes discarded.

Optimal culture conditions are known to those skilled in the art. All culture medium, supplements, plasticware and the like, must be endotoxin-free. Derivation of ES cell-like cultures has been described for cattle (Cibelli, et al., Theriogenology 47, 241 [1997]), hamster, (Doetschman, et al., Dev. Biol. 127, 224 [1988]), human (Thomson, et al., Science 282, 1145 [1998]) and rabbit (Schoonjans et al., Mol. Reprod. Dev. 45, 439 [1996]).

All patents and references cited herein are incorporated by way of reference. We further specifically incorporate by reference in its entirety Wakayama et al., Proceeding National Academy of Sciences, U.S.A., 96 (26):14984-14989 (Dec. 21, 1999).

1. A method for cloning an embryo comprising the steps of: (a) collecting the nucleus of a cultured cell; (b) microinjecting the nucleus of (a) or at least a portion of thereof that includes the chromosomes, into an enucleated oocyte to reconstitute the cell; and (c) allowing the reconstituted cell to develop embryonically.

2-4. (canceled)

5. The method of claim 1, wherein the cultured cell is an embryonic stem (ES) cell.

6-12. (canceled)

13. The method of claim 1, wherein the cultured cell is an embryonic germ (EG) cell.

14-17. (canceled)

18. The method of claim 1, wherein the cell of step (a) is genetically altered.

19-23. (canceled)

24. The method of claim 1, wherein the enucleated oocyte of step (b) is arrested at metaphase of the second meiotic division.

25. The method of claim 1, further comprising the step of activating the oocyte prior to, or during, or after the insertion of the cell nucleus or portion thereof.

26-27. (canceled)

28. The method of claim 25, wherein the activation step comprises electroactivation, or exposure to a chemical activating agent.

29. The method of claim 28, wherein the chemical activating agent is selected from the group consisting of ethyl alcohol, sperm cytoplasmic factors, oocyte receptor ligand peptide mimetics, pharmacological stimulators of Ca.sup.2+ release, Ca.sup.2+ ionophores, strontium ions, modulators of phosphoprotein signaling, inhibitors of protein synthesis, or combinations thereof.

30. The method of claim 28, wherein the chemical activating agent is selected from the group consisting of caffeine, the Ca.sup.2+ ionophore A23187, ethanol, 2-aminopurine, staurospurine, sphingosine, cyclohexamide, ionomycin, 6-dimethylaminopurine, soluble sperm-borne oocyte activating factor-I (SOAF-I.sub.S) or combinations thereof.

31. The method of claim 28, wherein the activating agent comprises Sr.sup.2+.

32. The method of claim 1, further comprising the step of disrupting microtubule and/or microfilament assembly in the oocyte for a time interval prior to or after insertion step (b).

33-38. (canceled)

39. A method for clonally deriving differentiated cells comprising the steps of: (a) collecting the nucleus of an ES cell; (b) microinjecting at least a portion of the ES cell nucleus that includes the chromosomes into an enucleated oocyte to form a reconstituted cell; (c) incubating the reconstituted cell for 0-6 hours prior to activation; (d) activating development of the reconstituted cell; and (e) allowing the reconstituted cell to develop.

40-46. (canceled)

47. (canceled)

48. A method for clonally deriving differentiated cells comprising the steps of: (a) collecting the nucleus of a cell; (b) microinjecting at least a portion of the cell nucleus of (a) that includes the chromosomes into an enucleated oocyte to form a reconstituted cell; (c) allowing the reconstituted cell to develop into a morula/blastocyst; (d) collecting an ES cell; (e) introducing the ES cell of (d) into the morula/blastocyst of (c); (f) allowing the reconstituted embryo of (e) to develop.

49-51. (canceled)

52. The method of claim 48, wherein the cell of step (a) is an ES cell derived from the same culture as the ES cell of step (d).

53. Differentiated cells produced by the method of claim 1.

54. An animal produced by the method of claim 1, whose nuclear chromosomes are derived from the nucleus of a cultured cell.

55-64. (canceled)

65. A method for modulating embryological development, comprising the steps of: (a) combining a nucleus of an ES cell with an enucleated oocyte to form a reconstituted cell; (b) inserting a reagent into the cytoplasm of the oocyte, prior to, during, or after the combining step; and (c) allowing the reagent-treated reconstituted cell to develop.

66-69. (canceled)

70. The method of claim 1, wherein the resulting embryo is dissociated and its cells allowed to differentiate into one or more cell lines.

71. The method of claim 1, wherein the cell lines are of cardiomyocytes, neuronal cells or hematopoietic cells.

72. Cells produced by the method of 70.
